(19) United States
(12) Patent Application Publication
(10) Pub. No.: US 2022/0162134 A1
(43) Pub. Date: May 26, 2022

(54) READILY ERECTABLE INSTALLATION RESPECTIVE KIT-OF-PARTS AND METHOD FOR PRODUCTION OF BIOGAS AND LIQUID FERTILIZER BY ANEROBIC DIGESTION ON INDUSTRIAL SCALE

(71) Applicant: HOME BIOGAS LTD, BEIT YANAI (IL)

(72) Inventors: OSHIK MOSHE EFRATI, BEIT YANAI (IL); EYAL GERZI, BET HASHITA (IL); OMRI COHEN, KORANIT (IL); BORIS STAVITSKY, TEL AVIV (IL); Avner PIPERBERG, Kadima-Zoran (IL); Yossi ROSENBLUM, Pardesiya (IL)

(73) Assignee: HOME BIOGAS LTD, BEIT YANAI (IL)

(21) Appl. No.: 17/671,018

(22) Filed: Feb. 14, 2022

Related U.S. Application Data
(63) Continuation-in-part of application No. 16/622,084, filed on Dec. 12, 2019.
Application No. 16/622,084 is a continuation of application No. 15/632,367, filed on Jun. 25, 2017, now Pat. No. 10,519,071.
Application No. 15/632,367 is a continuation-in-part of application No. 14/899,620, filed on Dec. 18, 2015, now Pat. No. 9,688,585.

(30) Foreign Application Priority Data
Jun. 18, 2013 (IB) 2013001272

(51) Int. Cl. C05F 9/02; C12M 1/107; C12M 1/00; C12P 5/02; C02F 3/28; C02F 11/04; C05F 17/964; C05F 17/40; C05F 17/907; C05F 17/914; C05F 17/979; C05F 17/986; C05G 5/20

(52) U.S. Cl. CPC C05F 9/02; C02F 2103/002; C12M 23/36; C12M 23/26; C12P 5/023; C02F 3/28; C02F 11/04; C05F 17/964; C05F 17/40; C05F 17/907; C05F 17/914; C05F 17/979; C05F 17/986; C05G 5/20; C12M 21/04

(57) ABSTRACT

A readily erectable installation for recycling organic waste into biogas and liquid fertilizer, on an industrial scale, implementing essentially anaerobic digestion processes, is described; the readily erectable installation includes: a cylindrically shaped assemblable enclosure and a pliant collapsible anaerobic digester, suspendable from the enclosure; a compact kit-of-parts for erecting the installation and respective method using the readily erectable installation for recycling organic waste into biogas and liquid fertilizer are described.

### CROSS-REFERENCE TO RELATED APPLICATIONS

The present application is a continuation-in-part of Ser. No. 16/622,084 filed Dec. 12, 2019, which is a 371 of PCT/IB2018/054643 filed Jun. 25, 2018 and continuation of Ser. No. 15/632,367 filed Jun. 25, 2017, which is a continuation-in-part of Ser. No. 14/899,620 filed Dec. 18, 2015, which is a national phase of international PCT application IB2013/061160 filed Dec. 19, 2013, claiming Paris convention priority from provisional application 61/916,246 filed Dec. 15, 2013 and international PCT application IB2013/001272 filed Jun. 18, 2013. The contents of the all the aforementioned cross-reference to related applications are incorporated herein in their entirety by this reference.

### TECHNICAL FIELD

In general, the present invention pertains to systems and methods of recycling organic waste and utilizing the products thereof. In particular, the invention relates to a readily erectable installation and respective kit-of-parts, implementable for recycling organic waste, implementing essentially anaerobic digestion processes, for production of biogas and liquid fertilizer on industrial scale.

### BACKGROUND ART

Household organic waste makes up a considerable percentage of total waste. This waste is typically thrown out with the rest of the garbage, requiring transport and space in dumps. Such waste is occasionally used for the purposes of producing compost, saving the transport and space requirements, as well as providing a source of rich soil. Hence improved system and methods for combined biogas and fertilizer production from such waste organic waste shall entail an environmental benefit.

Previous attempts include devices referred to as QuickQUBE, obtainable from QUBE RENEWABLES LIMITED at 21 Silver Street, Ottery St. Mary, EX11 1 DB Devon, UK, URL: www.quberenewables.co.uk/quickqube (https://web.archive.org/web/20191110151632/https://www.quberenewables.co.uk/quick qube). The QuickQUBE device a scaffolding framework is constructed is made from rigid columns and beams, which are typically welded. The anaerobic digester is attached by at least one rope to the columns or beams of this scaffolding framework.

Yet previous attempts include method and device, disclosed in US2010/233778, for generating biogas from organic materials having a biogas reactor which has a charging chamber for being charged with the organic materials and a backflow channel for an at least partial discharge of the organic materials from the biogas reactor.

US2015/126349 discloses a method for sealing and cutting of a flexible material for forming a flexible container comprising a product volume and at least one structural support volume can include feeding at least two flexible material into a sealing apparatus comprising a sealing surface and an opposed anvil surface; contacting a seam region of the at least two flexible material with the sealing surface to form a seal in the seam region and cut the seal to form a seam in a single unit operation.

It is further believed that the current state of the art is represented by U.S. Pat. Nos. 2,638,951, 5,429,437, 4,565,552, 5,924,461, 7,036,676, 7,186,339 and 9,688,585; European patent EP0045114; Chinese patents and utility models CN201575295, CN201400673, CN201915092 and CN202576409, as well as by international patent applications having publication numbers WO2011133023 and WO2012153256.

### BRIEF SUMMARY

U.S. Pat. No. 9,688,585 teaches a system and method of recycling organic waste into biogas, implementing an anaerobic digestion processes. The system of U.S. Pat. No. 9,688,585 includes a structural scaffolding and a pliable collapsible anaerobic digester. The aerobic digester in U.S. Pat. No. 9,688,585 includes at least one suspension tab, rendering the anaerobic digester suspendable from the structural scaffolding. A respective kit-of-parts is disclosed by U.S. Pat. No. 9,688,585 for assembling the aforementioned system.

In accordance with one aspect of the invention there are provided embodiments of a readily erectable installation for recycling organic waste on an industrial scale.

In accordance with another aspect of the invention there are provided embodiments of a method of readily erecting an installation for recycling organic waste on an industrial scale.

In accordance with yet another aspect of the invention there are provided embodiments of a method of recycling organic waste on an industrial scale, in a readily erectable installation therefor.

In accordance with some aspects and embodiments of the present invention, there is provided a respective kit-of-parts for assembling a readily erectable installation for production of biogas and liquid fertilizer on industrial scale, including a rigid assemblable enclosure made from a plurality of arcuate segments circumferentially assemblable in tandem around a longitudinal centerline of the rigid assemblable enclosure; a plurality of annular members assemblable in tandem along the longitudinal centerline of the rigid assemblable enclosure; a plurality of annular member connectors, configured to connect the annular members to form a cylindrical shape of the rigid enclosure.

In some embodiments, the anaerobic digester is attachable to the cylindrically shaped rigid assemblable enclosure by a plurality of fasteners.

In accordance with some aspects and embodiments of the present invention, a readily erectable installation for recycling organic waste on an industrial scale, the appliance includes: a pedestal whereon the readily erectable installation is mountable; a cylindrically shaped assemblable enclosure including: a plurality of arcuate segments circumferentially assemblable in tandem around a longitudinal centerline of the assemblable enclosure; a plurality of arcuate segment connectors, configured to connect the arcuate segments into an annular shape; a plurality of annular members, in which each one the annular members including a sub-assembly of the arcuate segments and the arcuate segment connectors, the annular members are assemblable in tandem along the longitudinal centerline of the assemblable enclosure; a plurality of annular member connectors, configured to connect the annular members to form a cylindrical shape of the assemblable enclosure; a suspendable hanger component including: a toroidally shaped structure; a plurality of structural elements, disposed on the toroidally shaped structure, configured for attachment of the toroidally shaped structure to interconnecting parts; a plurality of interconnecting parts, the interconnecting parts are configured to interconnect between the cylindrically shaped assemblable enclosure and the suspendable hanger component; an anaerobic digester including a sheet of pliable material, forming an essentially cylindrically closed structure; a plurality of fasteners attachable to an upper exterior portion of the anaerobic digester; in which each one of the plurality of fasteners including: at least one first portion, firmly attachable to the anaerobic digester; at least one second portion, operationally connectable to the first portion and the suspendable hanger component; in which each one of the plurality of fasteners is configured to assume: an open configuration, in which the portions are configured to receive the suspendable hanger component, and a closed configuration, in which the portions are configured to secure the suspendable hanger component; thereby rendering the anaerobic digester reversibly attachable to the suspendable hanger component.

In accordance with some aspects and embodiments of the present invention, a method of readily erecting an installation for recycling organic waste on an industrial scale, the appliance includes: constructing a pedestal whereon the installation is mountable; assembling a cylindrically shaped assemblable enclosure including: providing a plurality of arcuate segments; providing a plurality of arcuate segment connectors; assembling a plurality of annular member connectors by connecting the arcuate segments circumferentially in tandem around a longitudinal centerline of the cylindrically shaped assemblable enclosure; connecting the annular members in tandem around a longitudinal centerline of an assemblable enclosure; providing a suspendable hanger component including: providing a toroidally shaped structure; providing a plurality of structural elements, in which the toroidally shaped structure is disposed; providing a plurality of interconnecting parts; providing an anaerobic digester forming an essentially cylindrically closed structure; attaching a plurality of fasteners to an upper exterior portion of the anaerobic digester; in which attaching each one of the plurality of fasteners including: attaching at least one first portion to the anaerobic digester; connecting at least one second portion to the first portion and the suspendable hanger component; in which attaching each one of the plurality of fasteners including: receiving the suspendable hanger component, and securing the suspendable hanger component; in which switching between the open and closed configurations renders the anaerobic digester reversibly attachable to the suspendable hanger component.

In accordance with some aspects and embodiments of the present invention, a readily erectable installation for recycling organic waste on an industrial scale, the appliance includes: a pedestal whereon the readily erectable installation is mountable; a cylindrically shaped assemblable enclosure including: a plurality of arcuate segments circumferentially assemblable in tandem around a longitudinal centerline of the assemblable enclosure; a plurality of arcuate segment connectors, configured to connect the arcuate segments into an annular shape; a plurality of annular members, in which each one the annular members including a sub-assembly of the arcuate segments and the arcuate segment connectors, the annular members are assemblable in tandem along the longitudinal centerline of the assemblable enclosure; a plurality of annular member connectors, configured to connect the annular members to form a cylindrical shape of the assemblable enclosure; an anaerobic digester including a sheet of pliable material, forming an essentially cylindrically closed structure; a plurality of fasteners attachable to an upper exterior portion of the anaerobic digester; in which each one of the plurality of fasteners including: at least one first portion, attachable to the anaerobic digester; at least one second portion, operationally connectable to the first portion and a top portion of the cylindrically shaped assemblable enclosure; in which each one of the plurality of fasteners is configured to assume: an open conformation, in which the fastener is configured to receive the top portion of the cylindrically shaped assemblable enclosure, and a closed conformation, in which the fastener is configured to secure the top portion of the cylindrically shaped assemblable enclosure; thereby rendering the anaerobic digester reversibly attachable to the top portion of the cylindrically shaped assemblable enclosure.

In accordance with some aspects and embodiments of the present invention, a method of readily erecting an installation for recycling organic waste on an industrial scale, the appliance includes: constructing a pedestal whereon the installation is mountable; assembling a cylindrically shaped assemblable enclosure including: providing a plurality of arcuate segments; providing a plurality of arcuate segment connectors; assembling a plurality of annular member connectors by connecting the arcuate segments circumferentially in tandem around a longitudinal centerline of the cylindrically shaped assemblable enclosure; connecting the annular members in tandem around a longitudinal centerline of an assemblable enclosure; providing an anaerobic digester forming an essentially cylindrically closed structure; attaching a plurality of fasteners to an upper exterior portion of the anaerobic digester; in which attaching each one of the plurality of fasteners including: attaching at least one first portion to the anaerobic digester; connecting at least one second portion to the first portion and the cylindrically shaped assemblable enclosure; in which attaching each one of the plurality of fasteners including: receiving the cylindrically shaped assemblable enclosure, and securing the cylindrically shaped assemblable enclosure; in which switching between the open and closed configurations renders the anaerobic digester reversibly attachable to the cylindrically shaped assemblable enclosure.

In some embodiments, the pedestal is a platform.

In some embodiments, the toroidally shaped structure is a toroidally shaped cylindrical shell.

In some embodiments, the toroidally shaped structure is a curved tube which closes in on itself.

In some embodiments, the arcuate segments are arcuate corrugated metal sheet slabs.

In some embodiments, the interconnecting parts are is at least one of: a screw, bolt and nut, extended parallel coupler, fixing clamper, string, band, belt, loop and chain.

In some embodiments, the fastener is at least one of: of: a hook and loop fastener, string, buckle, snap connector, clip, strap, belt, chain, grommet, eyelet and tab.

In some embodiments, the anaerobic digester is fastened in a plurality of fastening points by a string.

In some embodiments, the plurality of the annular members, is pre-assembled from the plurality of the arcuate segments and the arcuate segment connectors, prior to assembling the plurality of the annular members in tandem along the longitudinal centerline of the assemblable enclosure.

### Definitions

The term torus (plural tori) and/or toroid, as referred to herein is to be construed as a surface of revolution generated by revolving a circle in three-dimensional space about an axis that is coplanar with the circle. If the axis of revolution does not touch the circle, the surface has a ring shape and is called a torus of revolution. If the axis of revolution is tangent to the circle, the surface is a horn torus. If the axis of revolution passes twice through the circle, the surface is a spindle torus. If the axis of revolution passes through the center of the circle, the surface is a degenerate torus, a sphere. If the revolved curve is not a circle, the surface is a related shape, a toroid.

The terms pliable or pliant, as referred to herein, are to be construed as having high tensile strength and capable of being efficiently elastically flexed or bent but not being resilient and incapable of being efficiently stretched or expanded. The term tensile or tensile strength, as referred to herein, is to be construed inter alia as a shortcut of the known term ultimate tensile strength, frequently represented acronym as UTS, meaning an intensive property of a material or structure to withstand loads tending to elongate, namely to resist tension, defined as the maximum stress that a material can withstand while been stretched or pulled before sustaining breaking, substantial deformation and/or necking before fracture, such as nylon, relating to essentially non-ductile materials, having UTS value ranging between about 600 and 1000 MPa or more, but not including rigid or stiff materials. In the present context, materials having rigidity modulus, otherwise referred to as the shear modulus, value of 4800 MPa or more are considered as rigid but not tensile, because such materials are incapable of being efficiently elastically flexed or bent. Stiff materials, such as steel, are defined as having rigidity modulus value well exceeding 4800 MPa.

The terms elastic or resilient, as referred to herein, are to be construed as having tensile strength lower than aforesaid tensile strength of pliable or pliant material and optionally being capable of efficiently stretching or expanding, relating inter alia to essentially ductile materials, having UTS value lesser than about 600 MPa.

The terms sheet or fabric, as referred to herein, is to be construed as including inter alia any spun-melt or non-woven fabrics.

The terms matching and/or matchable as referred to herein is to be construed as a cross-sectional area and/or shape of a component is equal or essentially similar to a cross-sectional area and/or shape of another component. It should be acknowledged that the component need only to be similar in the cross-sectional areas and/or shapes, to satisfy the term matching/matchable, so long as the cross-sectional areas can be mated or the combination will fit into and/or occupy essentially the same lateral space.

The term modular, as referred to herein, should be construed as a stand-alone unit. The term modular inter alia means a standardized unit that may be conveniently installed or deployed without significant impact to the environment. The term modular, however, doesn't necessarily means providing for ease of interchange or replacement. The term modular is optionally satisfied by providing for ease of at least onetime deployment or installation.

The term readily connectable, as referred to herein, should be construed as a standardized unit that may be conveniently connected to other components of the system. The term readily connectable, however, doesn't necessarily mean readily disconnectable or removable. The term readily connectable is optionally satisfied by providing for ease of at least onetime connection or coupling.

In the specification or claims herein, any term signifying an action or operation, such as: a verb, whether in base form or any tense, gerund or present/past participle, is not to be construed as necessarily to be actually performed but rather in a constructive manner, namely as to be performed merely optionally or potentially.

The term “substantially” as used herein is a broad term, and is to be given its ordinary and customary meaning to a person of ordinary skill in the art (and is not to be limited to a special or customized meaning), and refers without limitation to being largely but not necessarily entirely of that quantity or quality which is specified.

The term “essentially” means that the composition, method or structure may include additional ingredients, stages and or parts, but only if the additional ingredients, the stages and/or the parts do not materially alter the basic and new characteristics of the composition, method or structure claimed.

As used herein, the term “essentially” changes a specific meaning, meaning an interval of plus or minus ten percent (±10%). For any embodiments disclosed herein, any disclosure of a particular value, in some alternative embodiments, is to be understood as disclosing an interval approximately or about equal to that particular value (i.e., ±10%).

As used herein, the terms “about” or “approximately” modify a particular value, by referring to a range equal to the particular value, plus or minus twenty percent (+/−20%). For any of the embodiments, disclosed herein, any disclosure of a particular value, can, in various alternate embodiments, also be understood as a disclosure of a range equal to about that particular value (i.e. +/−20%).

As used herein, the term “or” is an inclusive “or” operator, equivalent to the term “and/or,” unless the context clearly dictates otherwise; whereas the term “and” as used herein is also the alternative operator equivalent to the term “and/or,” unless the context clearly dictates otherwise.

It should be understood, however, that neither the briefly synopsized summary nor particular definitions hereinabove are not to limit interpretation of the invention to the specific forms and examples but rather on the contrary are to cover all modifications, equivalents and alternatives falling within the scope of the invention.

### DESCRIPTION OF THE DRAWINGS

The present invention will be understood and appreciated more comprehensively from the following detailed description taken in conjunction with the appended drawings in which:

FIG. 1A is a front perspective view of a lightweight assemblable appliance, without the exterior enclosure;

FIG. 1B is a rear perspective view of the lightweight assemblable appliance, shown in FIG. 1A;

FIG. 2A is an isometric view of a lightweight or extremely lightweight assemblable appliance, supported and shaped by a pliant structured exoskeletal envelope;

FIG. 2B is a cross-sectional view of a lightweight or extremely lightweight assemblable appliance, supported and shaped by a pliant structured exoskeletal envelope;

FIG. 2C is an enlarged view showing details of exemplarily outlet assembly of the lightweight or extremely lightweight assemblable appliance, supported and shaped by a pliant structured exoskeletal envelope;

FIG. 2D is an enlarged cross-sectional view showing details of exemplarily outlet assembly of the lightweight or extremely lightweight assemblable appliance, supported and shaped by a pliant structured exoskeletal envelope;

FIG. 3 is an isometric view of a lightweight or extremely lightweight assemblable appliance, in a depleted or collapsed configuration;

FIG. 4 is an isometric view of a lightweight or extremely lightweight assemblable appliance, wherein the anaerobic digester is in a deployed or erected configuration, whereas the gas tank in a depleted or collapsed configuration;

FIG. 5 is an isometric view of a lightweight or extremely lightweight assemblable appliance, in a depleted or collapsed configuration;

FIG. 6 is an isometric view of a lightweight or extremely lightweight assemblable appliance, wherein the anaerobic digester is in a deployed or erected configuration, whereas the gas tank in a depleted or collapsed configuration;

FIG. 7 is an isometric view of a lightweight or extremely lightweight assemblable appliance, supported and shaped by a pliant structured exoskeletal envelope;

FIG. 8 is an isometric view of an extremely lightweight assemblable appliance, supported and shaped by a pliant structured exoskeletal envelope;

FIG. 9 is perspective view of a plunger handle;

FIG. 10A to 10C are perspective views of the constituents of the plunger handle;

FIG. 11A is an isometric view of a combined posterior assembly;

FIG. 11B is a cross-sectional view of a combined posterior assembly;

FIG. 12A is an isometric view of a readily erectable installation for recycling organic waste on an industrial scale, according to a preferred embodiment of the present invention;

FIG. 12B is an enlarged isometric view showing details of a readily erectable installation for recycling organic waste on an industrial scale, according to a preferred embodiment of the present invention;

FIG. 12C is an enlarged isometric view showing details of exemplarily fastener attached to an upper exterior portion of the anaerobic digester, according to a preferred embodiment of the present invention;

FIG. 13A is an isometric view of a readily erectable installation for recycling organic waste on an industrial scale, according to some preferred embodiment of the present invention;

FIG. 13B is an enlarged isometric view showing details of a readily erectable installation for recycling organic waste on an industrial scale, according to some preferred embodiment of the present invention;

FIG. 13C is an enlarged isometric view showing details of exemplarily fasteners attached to an upper exterior portion of the anaerobic digester, according to some preferred embodiment of the present invention;

FIG. 13D is an isometric view of the anaerobic digester, according to some preferred embodiments of the present invention;

FIG. 14 is a flowchart of the method of recycling organic waste on an industrial scale, according to some embodiments of the present invention.

While the invention is susceptible to various modifications and alternative forms, specific embodiments thereof have been shown merely by way of example in the drawings. The drawings are not necessarily complete and components are not essentially to scale; emphasis instead being placed upon clearly illustrating the principles underlying the present invention.

### DETAILED DISCLOSURE OF EMBODIMENTS

Illustrative embodiments of the invention are described below. In the interest of clarity, not all features of actual implementation are described in this specification. It will of course be appreciated that in the development of any such actual embodiment, numerous implementation-specific decisions must be made to achieve the developers' specific goals, such as compliance with technology- or business-related constraints, which may vary from one implementation to another. Moreover, it will be appreciated that the effort of such a development might be complex and time-consuming, but would nevertheless be a routine undertaking for those of ordinary skill in the art having the benefit of this disclosure.

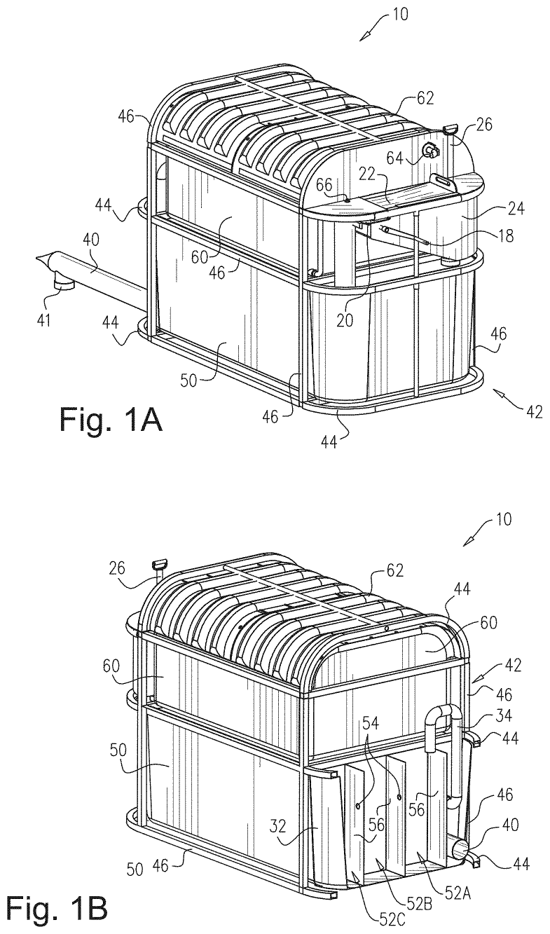

Reference is firstly made to FIGS. 1A and 1B, showing lightweight assemblable appliance 10. Lightweight assemblable appliance 10 forms an autonomic standalone unit, utilized for recycling organic waste into biogas and liquid fertilizer. Appliance 10 comprises anterior portion 14 and posterior portion 16. Anterior portion 14 and accommodates feeding sub-assembly comprising sink 24, grinder 20 and sink cover 22, as well as optionally fluid canister 28, or a fluid supply hose (not shown) disposed on top of sink 24, furnished with tap 30. Grinder 20 is typically driven either manually, for instance by the means of handle 18. Sink cover 22 is configured for conveniently feeding-in organic waste into grinder 20. The semiliquid mixture or slurry of ground organic matter and fluid is then fed into pliable collapsible anaerobic digester 50 through inlet pipe 27, which is connected to the outlet of sink 24. Inlet pipe 27 employed for feeding the semiliquid mixture or slurry of ground organic matter and fluid into anaerobic digester 50 is hermetically attached to anaerobic digester 50, so that the interior lumen of inlet pipe 27 forming a continuum with interior lumen of anaerobic digester 50. Inlet pipe 27 extends at least through a substantial portion of vertical dimension of anaerobic digester 50.

According to U.S. Pat. No. 9,688,585, multiple structural elements (not shown), such as flanges or pipe fittings, are attached to anaerobic digester 50 surfaces. In one embodiment, at least one inlet pipe 27 and or at least one slurry overflow outlet pipe 34 is/are connected to anaerobic digester 50 with such structural elements (not shown). In an embodiment, gas outlet pipe 59 is connected to anaerobic digester 50 with a structural member. In an embodiment, at least one sludge outlet pipe 40 is connected to anaerobic digester 50 with such a structural element.

According to U.S. Pat. No. 9,688,585, lightweight assemblable appliance 10 comprises posterior portion 16, which includes posterior compartment 32. Posterior compartment 32 forms an integral part of pliable collapsible anaerobic digester 50 or attached to anaerobic digester 50. Posterior compartment 32 may be divided by partitions 56, into sub-compartments 52A, 52B and 52C. Apertures 54 in partitions 56 interconnect between sub-compartments 52A to 52C. Sub-compartments 52A to 52C are configured to encompass overflow of liquid fertilizer or slurry resulting the digestion processes in anaerobic digester 50. Liquid fertilizer or slurry is optionally spilled over, from slurry overflow outlet pipe 34, having a siphon configuration, extending from a sidewall of anaerobic digester 50 into sub-compartment 52A. Sub-compartment 52C may include overflow outlet flange or pipe fitting 37, further furnished with nozzle 36. Sub-compartments 52A to 52C are optionally furnished with sealable drainage apertures 38, for conveniently emptying sub-compartments 52A to 52C upon opening of drainage apertures 38.

Posterior portion 16 further includes a sludge outlet draining pipe 40, extending from a bottom portion of a sidewall of anaerobic digester 50, configured for drainage of sludge and/or slurry resulting the digestion processes in anaerobic digester 50. Sludge outlet draining pipe 40 is preferably furnished with sealable cap or baffle 41, adapted for controllably opening/resealing sludge outlet draining pipe 40. Sludge outlet draining pipe 40 is pliable, allowing elevating the terminal portion thereof, thereby preventing the flow from anaerobic digester 50.

According to U.S. Pat. No. 9,688,585, lightweight assemblable appliance 10 comprises assemblable structural scaffolding 42. Structural scaffolding 42 comprises a plurality of arcuate structural members 44 and a plurality of linear structural members 46, interconnected by connectors 48. Structural scaffolding 42 is assemblable from a compact kit-of-parts comprising arcuate structural members 44, linear structural members 46 and connectors 48. Structural scaffolding 42 is characterized by the compactness of the kit-of-parts used for assembling it; thereby rendering assemblable appliance 10 suitable for shipment and transportation in a rather compact disassembled form. Structural scaffolding 42 comprises at least one structural member adapted for suspending pliable collapsible anaerobic digester 50, as elaborated infra.

According to U.S. Pat. No. 9,688,585, connectors 48 are embodied within terminal portions of structural members 44 and 46 and comprise an integral part of structural members 44 and 46. Structural members 44 and 46 thus interlock within each other, for instance by female and male endings of members 44 and 46; whereby multiple parts are connectable directly, without employing any individual connector 48 parts. Structural members 44 and 46 are profiles designed to provide increased bending strength. A couple of linear structural members 46 may be provided as a singular L-shaped member.

According to U.S. Pat. No. 9,688,585, anaerobic digester 50 is preferably made of at least one sheet of pliable material 51, defining an essentially closed rectangular parallelepiped shaped structure; thereby rendering anaerobic digester 50 pliable and collapsible. Anaerobic digester 50 is manufactured by welding of polymeric sheets. Therefore, anaerobic digester 50 is capable of assuming a collapsed or folded conformation, suitable for shipment and transportation in a rather compact folded form. Anaerobic digester 50 may be manufactured by welding and/or gluing segments polymeric sheets or by a means of molding, such as vacuum molding or blow molding.

According to U.S. Pat. No. 9,688,585, pliable collapsible anaerobic digester 50 shown in FIGS. 1A and 1B comprises elongated suspension tabs 58 attached along edges of anaerobic digester 50. Elongated suspension tabs 58 are attached to the surfaces of anaerobic digester 50. Structural members 46 are threaded into elongated suspension tabs 58, thereby rendering anaerobic digester 50 suspendable from structural scaffolding 42. Upon filling anaerobic digester 50 with the aforementioned semiliquid mixture or slurry of ground organic matter and fluid, while anaerobic digester 50 is suspended from structural scaffolding 42, stability is conferred to the structure of assemblable appliance 10 by the gravitational force exerted onto structural members 46 of scaffolding 42.

The suspension tabs, such as tabs 58, according to U.S. Pat. No. 9,688,585, mat embody a variety of shapes and/or structures as well as optionally include additional elements. The suspension tabs, such as tabs 58 may form an integral part of pliable collapsible anaerobic digester 50. Suspension tabs may include: a ring, an elongated sleeve, an abutment for attachment of another element, an element resembling a lifting ear. Anaerobic digester 50 may be suspended by straps and/or harness-like flexible structure (not shown), which are connected to structural scaffolding 42. In yet another embodiment, tab 58 comprises an extension of anaerobic digester 50 threaded into a slot in structural members 46.

Pliable collapsible anaerobic digester 50, according to U.S. Pat. No. 9,688,585, further comprises gas outlet pipe 59, hermetically attached to an upper face of digester 50 and extending upwardly therefrom. Baffle 70 is connected to gas outlet pipe 59, for controlling distribution of gas accumulated under positive pressure in pliable anaerobic digester 50 as a result of anaerobic digestion processes occurring therein. The gas distribution system may include safety valve 66, coupled to gas outlet pipe 59 and/or baffle 70 by conduit 72. Safety valve 66 is employed to release any excessive pressure of gas from anaerobic digester 50, upon exceeding a predetermined threshold. Gas distribution system further comprises conduit 74, coupling gas tank 60 to gas outlet pipe 59 and/or baffle 70.

According to U.S. Pat. No. 9,688,585, lightweight assemblable appliance 10 comprises a resilient gas tank or bladder 60, employed to accumulate the gas produced by the anaerobic digestion processes tacking place in anaerobic digester 50 under positive pressure for subsequent use. Resilient gas tank 60 is typically disposed on top of anaerobic digester 50. Resilient gas tank 60 may be detached from the structural scaffolding 42 while being connected to anaerobic digester 50 with a gas pipe 74. Resilient gas tank 60 can be made of at least one sheet of pliable and somewhat resilient material 61, defining an essentially closed structure; thereby rendering gas tank 60 collapsible as well as expandable or stretchable. Therefore resilient gas tank 60 capable of assuming a collapsed or depleted conformation, suitable for shipment and transportation in a rather compact folded form.

It is noted that resilient gas tank 60, according to U.S. Pat. No. 9,688,585, can assume a variety of shapes, inter alia cylindrical, semi-cylindrical and a somewhat rectangular shape, optionally having at least a convex upper face. Resilient gas tank 60 comprises inlet 67 coupled by conduit 74 to the gas distribution system. Resilient gas tank 60 further comprises gas outlet faucet 64, configured to allow conveniently utilizing the gas. Lightweight assemblable appliance 10 comprises array 62 of elongated and foldable ballast bags 80. Array 62 of ballast bags 80 is employed to exert gravitational force onto convex upper face of resilient gas tank 60, thereby contributing to the positive pressure of the gas inside gas tank 60 and rendering the gas inside gas tank 60 readily available for utilization. Ballast bags 80 are fillable with ballast substance, typically having a relatively high density or weight to volume ratio, such as sand. An array 62 of ballast bags 80 is capable of assuming an arcuate conformation, respectively conforming the surface of resilient gas tank 60. Array 62 of ballast bags 80 is capable of assuming a conformation, respectively conforming the shape of the top surface of pliable gas tank 60. Ballast bags 80 are disposed on foldable bands 82, which are optionally include apertures 86 along the edges thereof. Interconnecting strips 88 are threaded into apertures 86 to adjoin a plurality of foldable bands 82 in tandem. Fillable ballast bags 80 of array 62 are assuming a depleted conformation, suitable for shipment and transportation in a rather compact folded form. In some embodiment array 62 of ballast bags 80 is connected and/or forms an integral part of resilient gas tank 60.

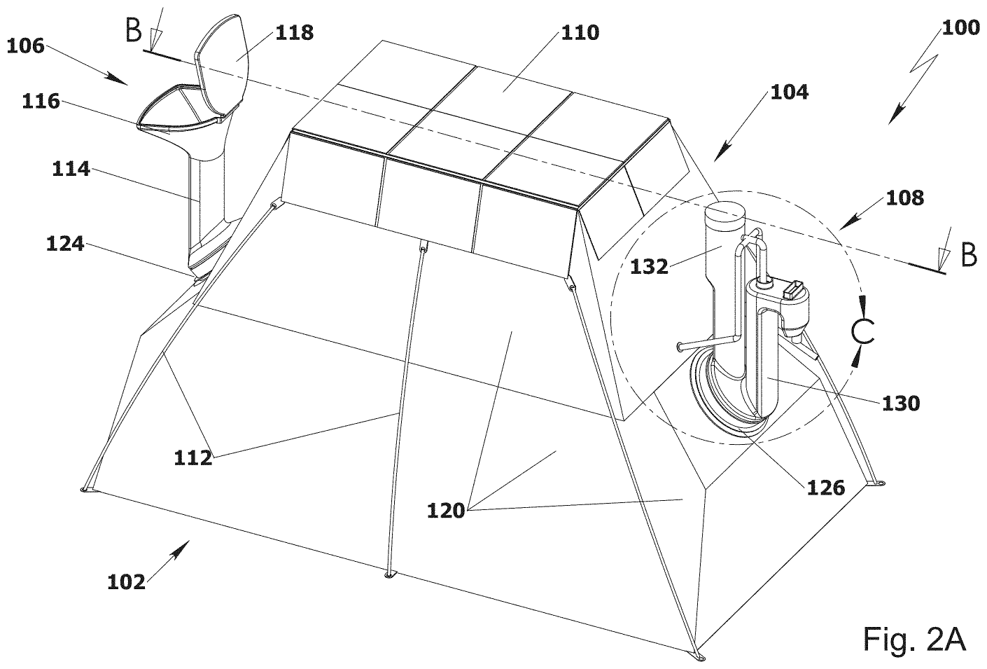

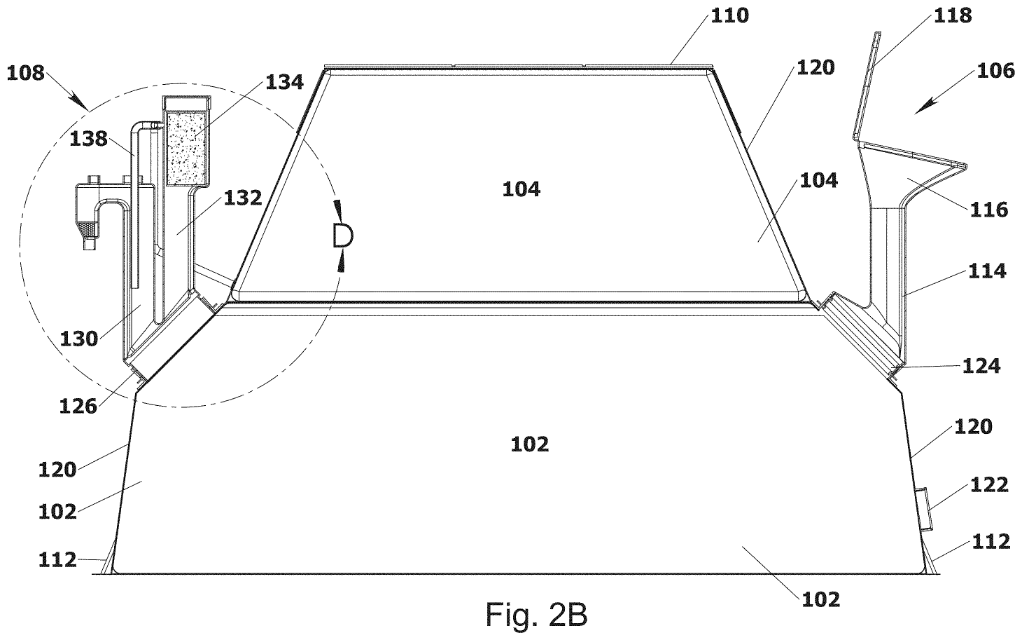

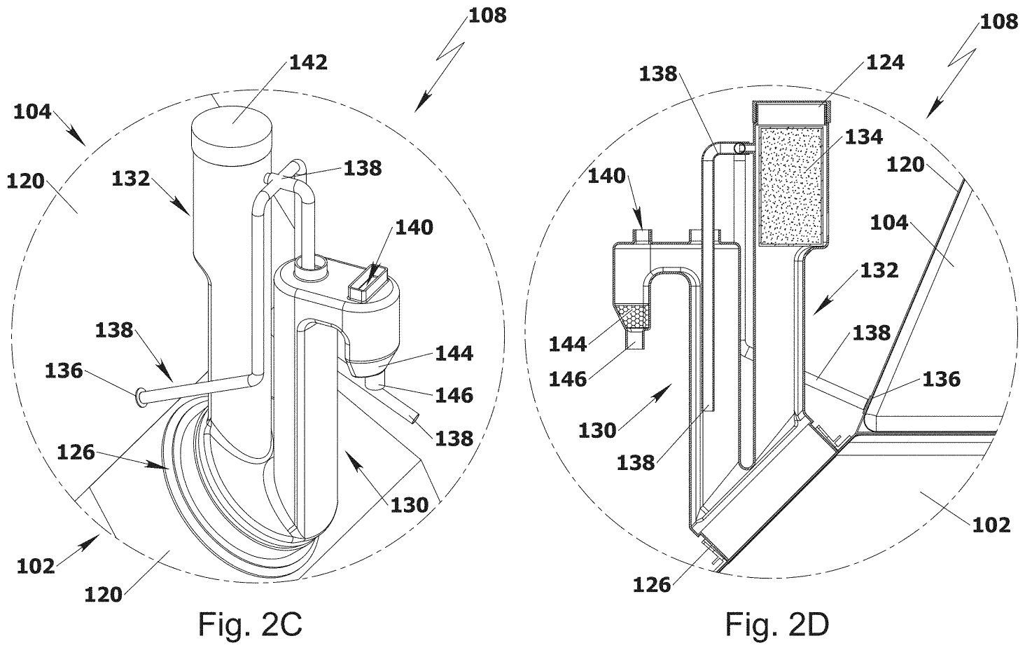

In some embodiments, reference is now made to FIGS. 2A and 2B, showing isometric cross-sectional views of lightweight or preferably extremely lightweight assemblable appliance 100, as well as to FIG. 2C to 2C, showing enlarged and cross-sectional enlarged views of outlet assembly 108. Appliance 100 comprises anaerobic digester 102 and gas tank 104. Digester 102 and tank 104 are made of elastic, resilient or pliable material.

Referring particularly to FIG. 2A to 2B, appliance 100 further comprises pliant structured exoskeletal envelope 120. Pliant structured exoskeletal envelope 120 defines a frusto-pyramidal shape, where anaerobic digester 102 is accommodated at the bottom portion of the pliant structured exoskeletal envelope 120, whereas gas tank 104 is accommodated at the top portion of the pliant structured exoskeletal envelope 120. Pliant structured exoskeletal envelope 120 confines digester 102 and tank 104 and thereby limits the expansion thereof.

Consequently, upon filling-up anaerobic digester 102 with semiliquid mixture or slurry or ground organic matter or any type of fluid for that matter, in a non-limiting manner including water, grey water and slurry overflow fluid, and/or upon forming positive pressure in gas tank 104, pliant structured exoskeletal envelope 120 is expanded and shaped-up by the pressure exerted from within by digester 102 and tank 104, to assume an erected or deployed confirmation, shown in FIGS. 2A and 2B. It is noted that the anaerobic digestion processes, occurring in pliable anaerobic digester 102, resulting a positive pressure in gas tank 104, mainly of methane gas. In some embodiments, organic matter optionally includes for animal droppings, which utilized by lightweight assemblable appliance 100, typically without grinding.

Upon filling-up anaerobic digester 102 with content and forming positive pressure in gas tank 104, pliant structured exoskeletal envelope 120 confers structural firmness to appliance 100, due to a normal counterforce to the force exerted by the faces of digester 102 and tank 104 on exoskeletal envelope 120, somewhat resembling the structural firmness of a wheel tire (not shown) conferred by the expansion of the inner tube (not shown). Pliant exoskeletal envelope 120 embodies a structured shape, configured to accommodate anaerobic digester 102 and gas tank 104, so as to limit their expansion to a maximal predetermined size.

Pliant exoskeletal envelope 120 is preferably made of woven or fibrous fabric, having high tensile strength and capable of being efficiently flexed or bent but incapable of being efficiently stretched or expanded. In some embodiments, pliant structured exoskeletal envelope 120 co-molded or welded with anaerobic digester 102 and/or gas tank 104, to form a monolithic constituent, in which anaerobic digester 102 and/or gas tank 104 are non-detachable pliant structured exoskeletal envelope 120. In other embodiments, pliant structured exoskeletal envelope 120 is an individual constituent distinct from anaerobic digester 102 and/or gas tank 104.

Anaerobic digester 102 comprises anterior flange 124, configured for connecting and mounting anterior inlet assembly 106, implementable for feeding semiliquid mixture, slurry, ground organic matter or a fluid, into anaerobic digester 102. Anterior flange 124 preferably comprises a feeding mechanism, such as a diaphragm or mitral valve (not shown), configured to sustain advancement of semiliquid mixture, slurry, ground organic matter or a fluid, fed into anaerobic digester 102, from anterior inlet assembly 106 but concurrently configured to prevent backflow of the contents from digester 102 into anterior inlet assembly.

Anaerobic digester 102 comprises posterior flange 126, configured for connecting and mounting posterior outlet assembly 108, implementable for draining grey water or overflow slurry fluid from anaerobic digester 102 as well as preferably for conducting the biogas produced by the anaerobic processes in digester 102 to gas tank 104 via conduit 138. Anaerobic digester 102 optionally comprises anterior opening with removable plug 124, configured for occasionally depleting the sludge that may accumulate in digester 102, as a part of maintenance of lightweight assemblable appliance 100. It is, however, noted that anterior opening with removable plug 124, configured for depleting the sludge from digester 102, is merely optional, whereas in some embodiments there is no dedicated opening for depleting the sludge from digester 102.

In order to yet further facilitate an increased pressure inside gas tank 104, appliance 100 further comprises at least one pressure forming mechanism. Embodiments of pressure forming mechanisms in a non-limiting manner include gravitational and/or bias driven devices. Examples of gravitational devices include array of ballast bags or pockets 110, fillable with ballast substance (not shown), configured to facilitate increased pressure by exerting gravitational force onto inside gas tank 104.

Examples of bias driven devices include elastic tension straps 112, comprising an elastomeric material, connected to respective elements attached to the bottom of appliance 100, configured to facilitate increased pressure by exerting tensile strain force onto inside gas tank 104. Notably a combination of gravitational and/or bias driven devices is equally contemplated by this disclosure.

Referring particularly to FIGS. 2C and 2D, anterior inlet assembly 106 comprises feeding conduit 114, which is optionally made of solid, stiff or firm material, capable of supporting its own weight. Feeding conduit 114 terminates with inlet funnel 116, coverable by pivoting and preferably biased lid 118. In some examples feeding conduit 114 is made of flexible or pliant material, incapable of supporting its own weight, in such cases inlet funnel 116 is supported by a bipod (not shown) structure.

Posterior outlet assembly 108 comprises slurry overflow outlet portion 130 and gas ducting portion 132. Slurry overflow outlet portion 130 comprises chlorinator 144, chlorinator filling port 140 and slurry overflow nozzle 146. Slurry overflow nozzle 146 is disposed downstream to chlorinator 144, so that any overflow of slurry from digester 102 to outlet portion 130 passes through chlorinator 144, thereby rendering the fluids outflowing from slurry nozzle 146 non-virulent and biologically safe for the environment or use for irrigation in agriculture.

Gas ducting portion 132 of posterior outlet assembly 108 further comprises biogas filter 134, configured for absorbing sulfurous compounds from the biogas produced in anaerobic digester 102. The biogas filter 134 optionally comprises activated carbon or activated charcoal, which is replaceable from the top opening covered by plug 142. Gas infiltrating through biogas filter 134 is supplied into gas piping 138. Gas piping 138 extends from gas ducting portion 132 of posterior outlet assembly 108 to gas inlet 136 of gas tank 104. Gas piping 138 further extends to a gas-powered consuming appliance (not shown). Gas piping 138 further optionally extends into slurry overflow outlet portion 130. Gas piping further 138 optionally comprises check valves, configured to conduct the biogas only in one direction, and/or safety valves, configured to conduct the biogas only above a predetermined pressure threshold.

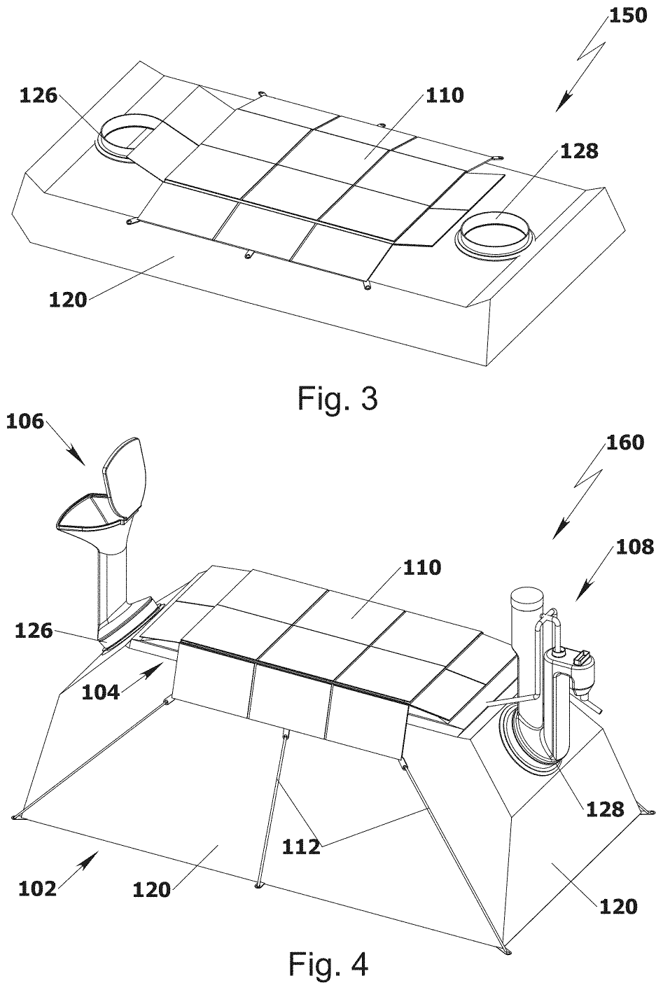

Reference is now made to FIG. 3, showing the lightweight or preferably extremely lightweight assemblable appliance in folded or collapsed conformation 150. Lightweight assemblable appliance in folded conformation 150 is configured for assuming a compact size. Lightweight assemblable appliance in folded conformation 150 is typically folded yet further laterally or rolled up to assume a compact size (not shown), configured for shipment and transportation at the back seat of an economy car and/or by air cargo.

Reference is now made to FIG. 4, showing the lightweight or preferably extremely lightweight assemblable appliance in a partially erected or deployed conformation 160. Lightweight assemblable appliance assumes a partially erected or deployed conformation 160 upon filling-up anaerobic digester 102 with liquid. Gas tank 104 of lightweight assemblable appliance in a partially erected or deployed conformation 160 is empty of biogas. With the progression of anaerobic processes in anaerobic digester 102, biogas filling-up gas tank 104 and lightweight assemblable appliance assumes completely erected or deployed conformation 100, shown in FIGS. 2A and 2B.

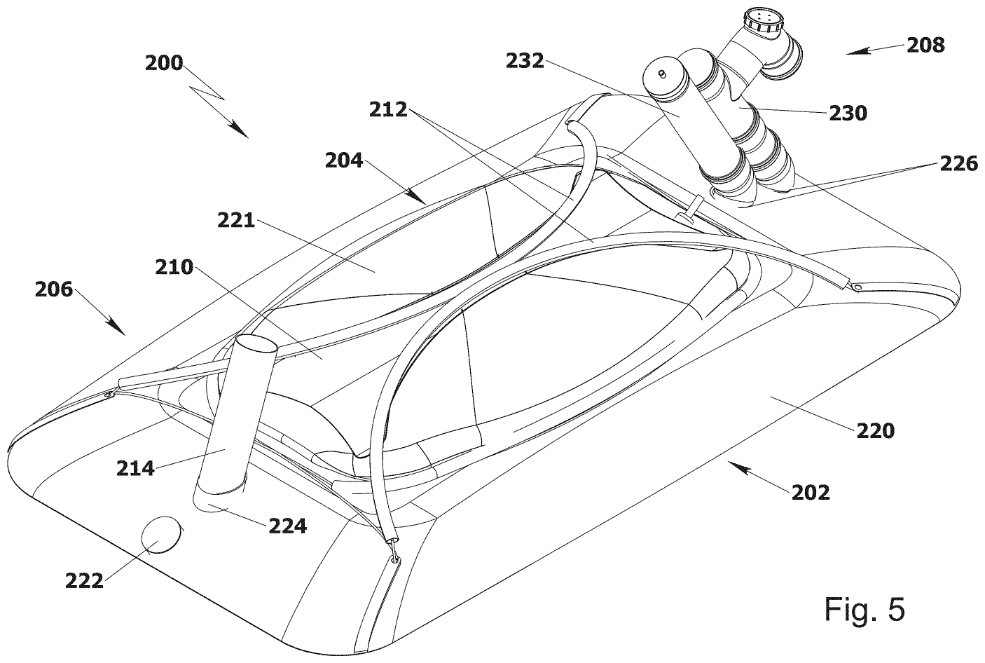

Reference is now made to FIGS. 5 to 7, showing isometric views of lightweight or preferably extremely lightweight assemblable appliance 200. Appliance 200 comprises anaerobic digester 202 and gas tank 204. Digester 202 and tank 204 are made of elastic, resilient or pliable material.

Appliance 200 further comprises pliant structured exoskeletal envelope 220 for anaerobic digester 202 and pliant structured exoskeletal envelope 221 for gas tank 204. Pliant structured exoskeletal envelops 220 defines a frusto-pyramidal shape, where anaerobic digester 202 is accommodated, whereas pliant structured exoskeletal envelope 221 defines a frusto-pyramidal shape, where gas tank 104 is accommodated. Pliant structured exoskeletal envelopes 220 and 221 respectively confine digester 202 and tank 204, thereby limiting the expansion thereof.

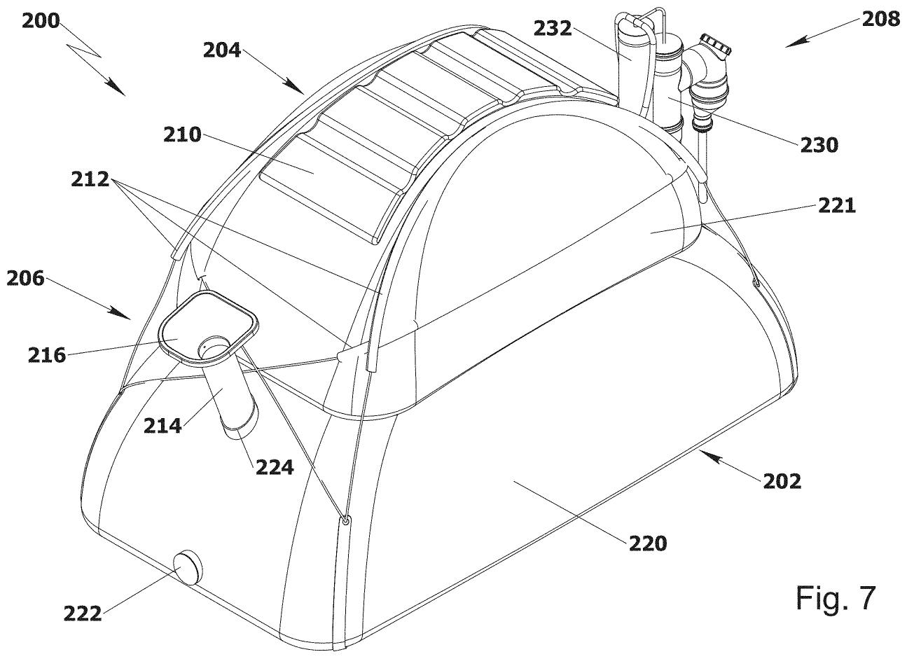

Consequently, upon filling-up anaerobic digester 202 with semiliquid mixture or slurry or ground organic matter or any type of fluid for that matter, in a non-limiting manner including water, grey water and slurry overflow fluid, and/or upon forming positive pressure in gas tank 204, pliant structured exoskeletal envelopes 220 and 221 are expanded and shaped-up by the pressure exerted from within by digester 202 and tank 204, to assume an erected or deployed confirmation, shown in FIG. 7. It is noted that the anaerobic digestion processes, occurring in pliable anaerobic digester 202, resulting a positive pressure in gas tank 204, mainly of methane gas. In some embodiments, organic matter optionally includes for animal droppings, which utilized by lightweight assemblable appliance 200, typically without grinding.

Upon filling-up anaerobic digester 202 with content and forming positive pressure in gas tank 204, pliant structured exoskeletal envelope 220 and 221 confer structural firmness to appliance 200, due to a normal counterforce to the force exerted by the faces of digester 202 and tank 204 on exoskeletal envelopes 220 and 221, somewhat resembling the structural firmness of a wheel tire (not shown) conferred by the expansion of the inner tube (not shown). Pliant exoskeletal envelopes 220 and 221 embody structured shapes, configured to accommodate anaerobic digester 202 and gas tank 204, so as to limit their expansion to a maximal predetermined size.

Pliant exoskeletal envelopes 220 and 221 are preferably made of woven or fibrous fabric, having high tensile strength and capable of being efficiently flexed or bent but incapable of being efficiently stretched or expanded. In some embodiments, pliant structured exoskeletal envelopes 220 and 221 are co-molded or welded with anaerobic digester 202 and/or gas tank 204, to form a monolithic constituent, in which anaerobic digester 202 and/or gas tank 204 are non-detachable pliant structured exoskeletal envelopes 220 and 221.

In some embodiments, pliant structured exoskeletal envelopes 220 and 221 are co-molded or welded with anaerobic digester 202 and/or gas tank 204, so that envelopes 220 and 221 as well as digester 202 and/or gas tank 204 comprise composite materials. A preferred instance of composite material used for manufacture the complex of exoskeletal envelope 220 and anaerobic digester 202 is a multilayered PVC sheet with embedded nylon or other polymeric pliable fibers.

In some embodiments, pliant structured exoskeletal envelopes 220 and 221 are a unified singular pliant structured exoskeletal envelope, such as envelope 120 shown in FIGS. 2 to 4. In other embodiments, pliant structured exoskeletal envelopes 220 and 221 are individual constituents distinct from anaerobic digester 202 and/or gas tank 204.

Anaerobic digester 202 comprises anterior flange 224, configured for connecting and mounting anterior inlet assembly 206, implementable for feeding semiliquid mixture, slurry, ground organic matter or a fluid, into anaerobic digester 202. Anterior flange 224 preferably comprises a feeding mechanism, such as a diaphragm or mitral valve (not shown), configured to sustain advancement of semiliquid mixture, slurry, ground organic matter or a fluid, fed into anaerobic digester 202, from anterior inlet assembly 206 but concurrently configured to prevent backflow of the contents from digester 202 into anterior inlet assembly.

Anaerobic digester 202 comprises posterior flanges 226, configured for connecting and mounting posterior outlet assembly 208, implementable for draining grey water or overflow slurry fluid from anaerobic digester 202 as well as for conducting the biogas produced by the anaerobic processes in digester 202 to gas tank 204. Anaerobic digester 202 comprises anterior opening 222 with removable plug, configured for occasionally depleting the sludge that may accumulate in digester 202, as a part of maintenance of lightweight assemblable appliance 200.

In order to yet further facilitate an increased pressure inside gas tank 204, appliance 200 further comprises at least one pressure forming mechanism. Embodiments of pressure forming mechanisms in a non-limiting manner include gravitational and/or bias driven devices. Examples of gravitational devices include array of ballast bags or pockets 210, fillable with ballast substance (not shown), configured to facilitate increased pressure by exerting a gravitational force onto gas tank 204.

Examples of bias driven devices include elastic tension straps 212, comprising an elastomeric material, connected to respective elements attached to the bottom of appliance 200, configured to facilitate increased pressure by exerting tensile strain force onto inside gas tank 204. Notably a combination of gravitational and/or bias driven devices is equally contemplated by this disclosure.

Anterior inlet assembly 206 comprises feeding conduit 214, which is optionally made of solid, stiff or firm material, capable of supporting its own weight. Feeding conduit 214 terminates with inlet funnel 216, preferably coverable by pivoting and preferably biased lid (not shown). In some examples feeding conduit 214 is made of flexible or pliant material, incapable of supporting its own weight, in such cases inlet funnel 216 is supported by a bipod (not shown) structure.

Posterior outlet assembly 208 comprises slurry overflow outlet portion 230 and gas ducting portion 232. Slurry overflow outlet portion 230 preferably comprises a chlorinator (not shown) with a chlorinator filling port and a slurry overflow nozzle. The slurry overflow nozzle is disposed downstream to the chlorinator (not shown), so that any overflow of slurry from digester 202 to outlet portion 230 passes through the chlorinator (not shown), thereby rendering the fluids outflowing from the slurry nozzle non-virulent and biologically safe for the environment or use for irrigation in agriculture.

Gas ducting portion 232 of posterior outlet assembly 208 further comprises biogas filter (not shown), configured for absorbing sulfurous compounds from the biogas produced in anaerobic digester 202. The biogas filter (not shown) optionally comprises activated carbon or activated charcoal, which is replaceable from the top opening covered by a plug (not shown). Gas infiltrating through a biogas filter (not shown) is supplied into gas piping (not shown). The gas piping (not shown) extends from gas ducting portion 232 of posterior outlet assembly 208 to the gas inlet (not shown) of gas tank 204. The gas piping (not shown) further extends to a gas-powered consuming appliance (not shown). The gas piping (not shown) further optionally extends into slurry overflow outlet portion 230. The gas piping further (not shown) optionally comprises check valves, configured to conduct the biogas only in one direction, and/or safety valves, configured to conduct the biogas only above a predetermined pressure threshold.

Reference is now made to FIG. 5, showing the lightweight or preferably extremely lightweight assemblable appliance 200 in folded or collapsed conformation. Lightweight assemblable appliance 200 in folded conformation, shown in FIG. 5, is configured for assuming a compact size. Lightweight assemblable appliance 200, shown in FIG. 5, in folded conformation is typically folded yet further laterally or rolled up to assume a compact size (not shown), configured for shipment and transportation at the back seat of an economy car and/or by air cargo.

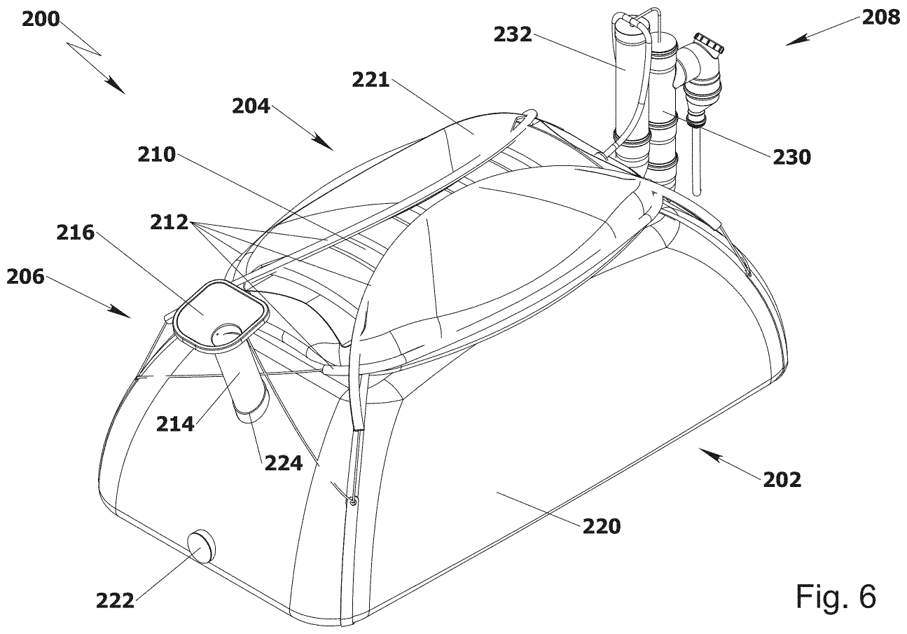

Reference is now made to FIG. 6, showing the lightweight or preferably extremely lightweight assemblable appliance 200 in a partially erected or deployed conformation. Lightweight assemblable appliance assumes a partially erected or deployed conformation, shown in FIG. 6, upon filling-up anaerobic digester 202 with liquid. Gas tank 204 of lightweight assemblable appliance 200 in a partially erected or deployed conformation, shown in FIG. 6, is empty of biogas. With the progression of anaerobic processes in anaerobic digester 202, biogas filling-up gas tank 204 and lightweight assemblable appliance 200 assumes completely erected or deployed conformation, shown in FIG. 7.

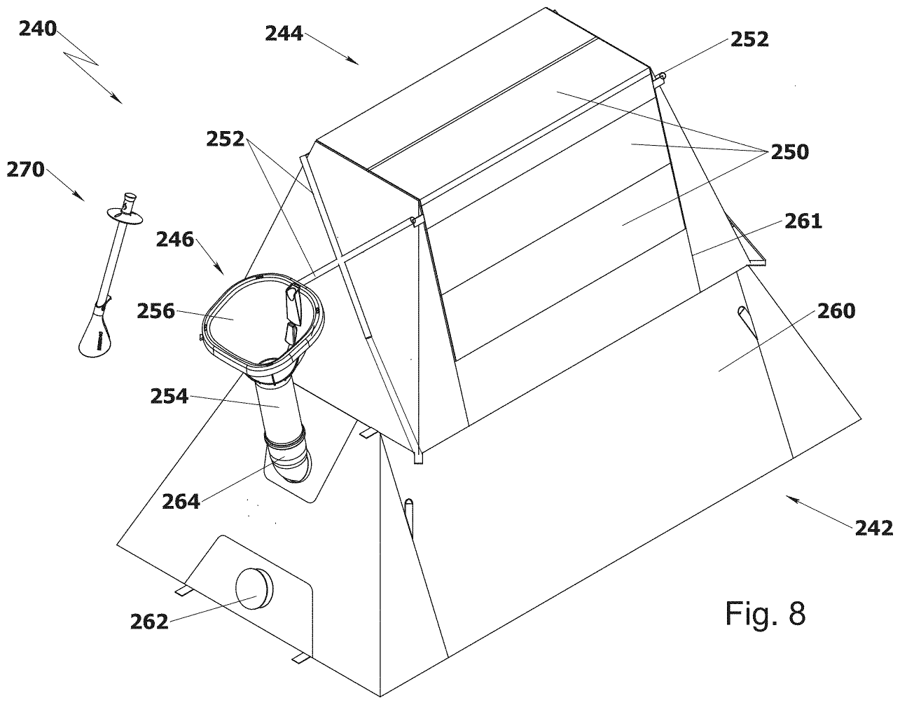

Reference is now made to FIG. 8, showing another embodiment of extremely lightweight assemblable appliance 240. Appliance 240 comprises anaerobic digester 242 supported and shaped by pliant structured exoskeletal envelope 260 and gas tank 244 supported and shaped by pliant structured exoskeletal envelope 261. Pliant structured exoskeletal envelopes 260 and 261 respectively confine digester 242 and tank 244, thereby limiting the expansion thereof.

In order to yet further facilitate an increased pressure inside gas tank 244, appliance 240 further comprises at least one pressure forming mechanism, such as array of ballast bags or pockets 250, fillable with ballast substance (not shown), and/or elastic tension straps 252, comprising an elastomeric material, connected to respective elements attached to the bottom of appliance 240, configured to facilitate increased pressure by exerting tensile strain force onto inside gas tank 244. Notably tension straps 252 are attached to the bottom portion of pliant structured exoskeletal envelope 261 enclosing gas tank 244; thereby exerting the tensile strain force only onto gas tank 244.

Anaerobic digester 242 comprises anterior flange 264, configured for connecting and mounting anterior inlet assembly 246, implementable for feeding semiliquid mixture, slurry, ground organic matter or a fluid, into anaerobic digester 242. Anterior inlet assembly 246 comprises feeding conduit 254, typically made of solid, stiff or firm material. Feeding conduit 254 terminates with inlet funnel 256. Anterior inlet assembly 246 preferably comprises a feeding mechanism, such as plunger 270, configured to sustain advancement of semiliquid mixture, slurry, ground organic matter or a fluid, fed into anaerobic digester 242, from anterior inlet assembly 246.

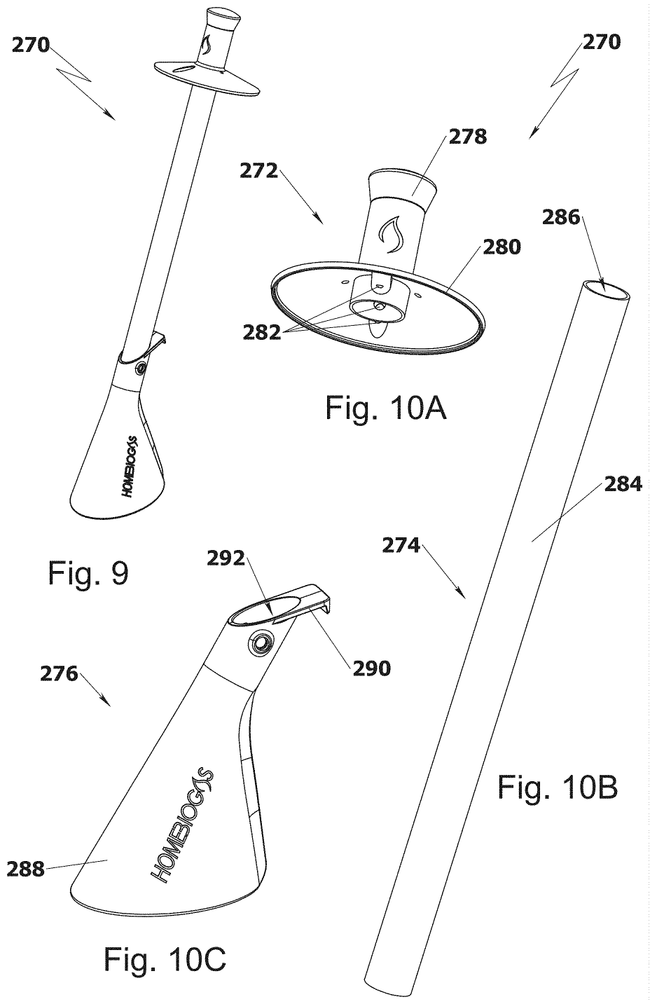

Reference is now made to FIG. 9 showing plunger handle 270 in greater details as well as to FIG. 10A-C showing constituents thereof. Plunger handle 270 comprises handle 272 shown in FIG. 10A, shaft 274 shown in FIG. 10B and terminal part 276 shown in FIG. 10C. Handle 272 comprises proximal portion 278, configured for manual gripping. Handle 272 further comprises conduits 282, configured for conduct air from anterior inlet assembly 246, thereby avoiding back splash from inlet assembly 246 and/or anaerobic digester 242, upon advancing a semiliquid mixture, slurry, ground organic matter or fluid, fed into anaerobic digester 142.

Handle 272 comprises plug 280, configured blocking the opening of feeding conduit 254 at the bottom of inlet funnel 256. Shaft 274 comprises essentially hollow firm pipe 284, defining interior lumen 284, configured for conducting the air from anterior inlet assembly 246, upon advancing the semiliquid mixture or fluid into anaerobic digester 242. Terminal part 276, shown in FIG. 10C, comprises a frusto-conical mitral skirt 288, configured for advancing the semiliquid mixture or fluid into feeding conduit 254, while concurrently preventing backflow of the contents from digester 242 into anterior inlet assembly 246. The top portion of terminal part 276 embodies hanging hook 290 configured for hanging plunger handle 270 from inlet funnel 256 and defines air inlet, configured for conducting the air from anterior inlet assembly 246 into pipe 284, upon advancing the semiliquid mixture or fluid into anaerobic digester 242.

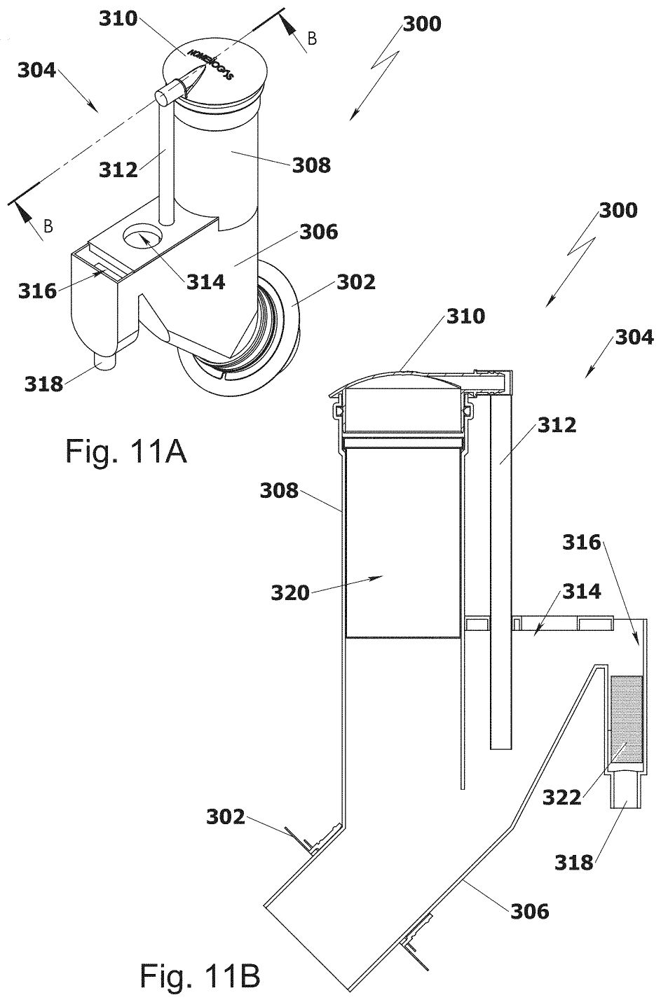

Reference is now made to FIGS. 11A and 11B, showing an embodiment of combined posterior assembly 300. Posterior assembly 300 is mounted onto posterior outlet flange 302, attached to anaerobic digester (not shown), such as anaerobic digester 242 shown in FIG. 8. Posterior assembly 300 comprises slurry overflow outlet portion 306 and gas ducting portion 308. Slurry overflow outlet portion 306 comprises chlorinator 322, chlorinator filling port 316 and slurry overflow nozzle 318. Slurry overflow nozzle 318 is disposed downstream to chlorinator 322. Slurry overflow outlet portion 306 further comprises surplus overflow opening 314, for any excessive slurry overflow that is not drained via nozzle 318.

Gas ducting portion 308 of posterior outlet assembly 300 further comprises gas filter lumen 320. Gas filter lumen 320 configured to contain a substance, such as activated charcoal (not shown), absorbing sulfurous compounds from the biogas, which is replaceable from the top opening covered by plug 310. Gas piping 312 preferably extends into slurry overflow outlet portion 306.

Wherever in the specification hereinabove and in claims hereunder it is noted that the pliable collapsible anaerobic digester, such as digesters 50, 102, 202 or 242, including or comprising an inlet pipe, gas outlet pipe, slurry overflow outlet pipe or sludge outlet draining pipe—it should be construed that the pliable collapsible anaerobic digester includes or comprises merely a preparation on the surface thereof and/or inside the wall thereof as well as an additional element for relatively easily mounting and/or attaching an inlet pipe, gas outlet pipe, slurry overflow outlet pipe or sludge outlet draining pipe thereto, whereas the inlet pipe, gas outlet pipe, slurry overflow outlet pipe or sludge outlet draining pipe are not provided or attached to the digester.

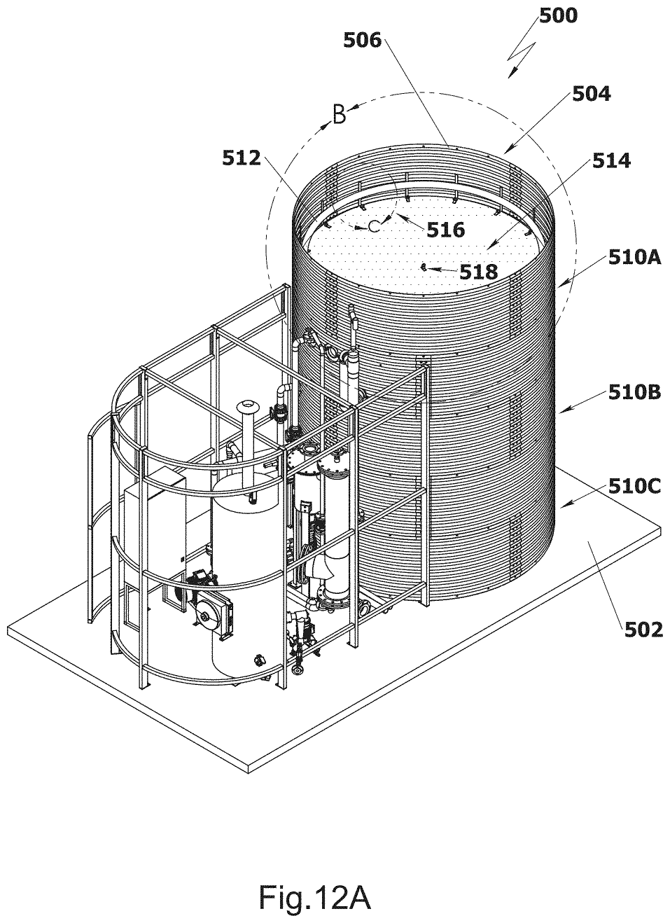

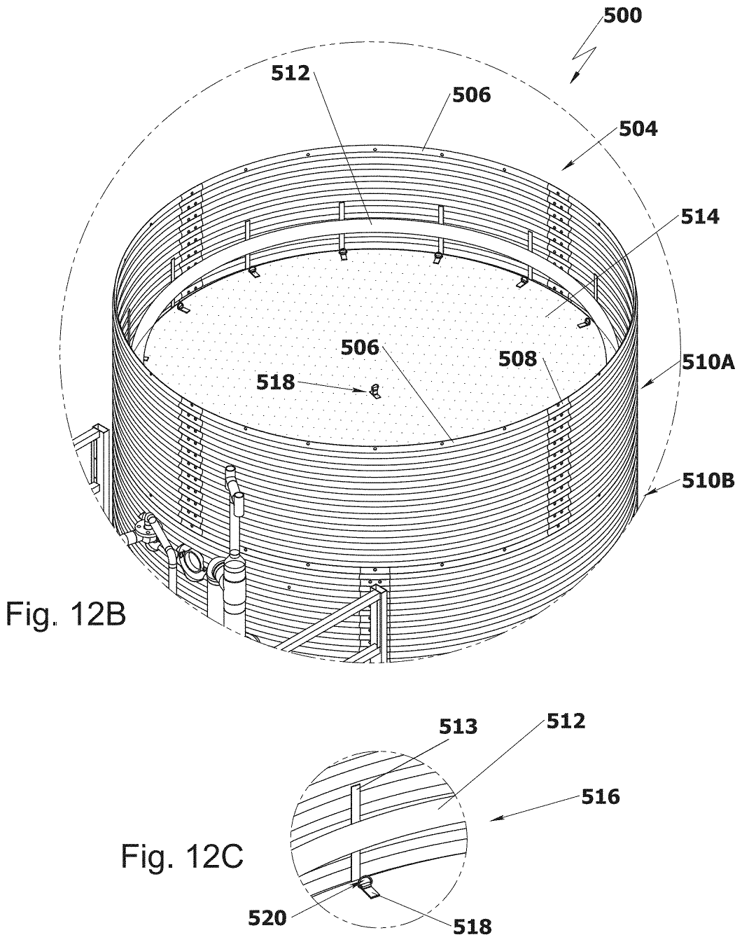

In accordance with some preferred embodiments of the present invention, reference is now made to FIG. 12A to 12C, showing an isometric view of a readily erectable installation 500 for recycling organic waste on an industrial scale with a hanger component, as well as a couple of enlarged views thereof. Erectable installation 500 for recycling organic waste on an industrial scale of the embodiment of FIG. 12A to 12C illustrates various features that may be interchangeable with elements of any other embodiment described in the specification.

Installation 500 comprises pedestal 502, whereon readily erectable installation 500 is mountable. In some examples, pedestal 502 is a circular or rectangular platform, for instance made of concrete or wood, having a sufficient circumference or perimeter to accommodate the components of installation 500 and bear their respective weight. The platform of pedestal 502 forms a horizontal surface used as a support for the mounting of installation 500 thereon. In some examples, where readily erectable installation 500 is installable on existing floor, pedestal 502 onto which installation 500 is mountable, is portion of such existing floor, having essentially flat top horizontal surface.

In some embodiments, installation 500 comprises cylindrically shaped assemblable enclosure 504. In some examples, cylindrically shaped assemblable enclosure 504 is attachable to pedestal 502 by screws and/or bolts and/or brackets and/or fasteners, thereby affixing installation 500 to the top horizontal surface pedestal 502. In some embodiments, cylindrically shaped assemblable enclosure 504 comprises a plurality of annular members 510A, 5108 and 510C.

In some embodiments, annular members 510A, 5108 and 510C are assemblable in tandem along the longitudinal centerline of assemblable enclosure 504. In some examples, each pair of adjacent annular members, such as the pairs of annular members 510A and 5108 and annular members 5108 and 510C, are respectively connected to each other by plurality of annular member connectors, typically about the circumference thereof, to form a cylindrical shape of assemblable enclosure 504. In some examples, annular members connectors include: a fixing clamp, a segment attach fitting, a round steel clamps, a steel pipe clamp, rivets, bolts and nuts, etc.

In some embodiments, each one of the plurality of annular members 510A, 510B and 510C comprises a sub-assembly of arcuate segments 506 connected by arcuate segments connectors 508. In some embodiments, a plurality of arcuate segments 506 are circumferentially assemblable around a longitudinal centerline of cylindrically shaped assemblable enclosure 504 to form the plurality annular members 510A, 510B and 510C. Each pair of adjacent arcuate segments 506 are connectable by arcuate segment connectors 508, typically at the straight terminal portions thereof, thereby forming an annular shape of members 510A, 510B and 510C. In some examples, arcuate segments connectors 508 in a non-limiting manner include: a fixing clamp, a segment attach fitting, a round steel clamps, a steel pipe clamp, rivets, bolts and nuts, etc.

On the contrary, in prior art in general and particularly in the above-referenced QuickQUBE system, a polygonal rigid frame is constructed first from solid columns and beams. The prior art digester is then attached by ropes to the columns of this polygonal rigid frame. The prior art polygonal rigid frame, such as in the above-referenced QuickQUBE system, to which the anaerobic digester is attached, is not readily erectable but requires preceding construction for deployment and erecting of the system. Contradistinctively to the prior art, cylindrically shaped assemblable enclosure 504 that is formed by the plurality annular members 510A, 510B and 510C, which in turn are assemblable from arcuate segments 506, contemplated by the present invention and described in the specification hereinabove is readily erectable and does not require preceding construction other than of flat pedestal 502, for deployment and erecting of the system.

In some embodiments, installation 500 comprises suspendable hanger component 512. In some examples, suspendable hanger component 512 comprises a toroidally shaped structure. In some examples, suspendable hanger component 512 is a long curved tube or rod which closes in on itself, for instance by welding, thereby forming a toroidally shaped structure. In some embodiments, suspendable hanger component 512 is suspended from assemblable enclosure 504. Furthermore, anaerobic digester 514 is hanged from and/or on suspendable hanger component 512, as will be elaborated hereunder.

In some embodiments, suspendable hanger component 512 comprises plurality of structural elements. The plurality of structural elements is disposed on toroidally shaped structure of suspendable hanger component 512. The plurality of structural elements of suspendable hanger component 512 are configured for connecting toroidally shaped structure of suspendable hanger component 512 to interconnecting parts 513. In some embodiments, cylindrically shaped assemblable enclosure 504 comprises a plurality of respective structural elements. The plurality of respective structural elements of cylindrically shaped assemblable enclosure 504 are configured for connecting cylindrically shaped assemblable enclosure 504 to the aforementioned interconnecting parts 513.

In some examples, the plurality of structural elements of suspendable hanger component 512 and the plurality of respective structural elements of cylindrically shaped assemblable enclosure 504 are plurality of apertures, blind holes, grommets, eyelets, hooks, tabs, etc. In some embodiments, opposite to each structural element of toroidally shaped structure of suspendable hanger component 512 there is disposed a respective structural element of the plurality of respective structural elements of cylindrically shaped assemblable enclosure 504, typically at the upper portion of annular member 510A of cylindrically shaped assemblable enclosure 504.

In some embodiments, installation 500 comprises a plurality of interconnecting parts 513. The plurality of interconnecting parts 513 is configured to interconnect between cylindrically shaped assemblable enclosure 504 and suspendable hanger component 512. In some examples, interconnecting part 513 in a non-limiting manner include: a screw, bolt and nut, extended parallel coupler, fixing clamper, string, band, belt, loop, chain, etc.

In some examples, into each structural element of toroidally shaped structure of suspendable hanger component 512 is inserted a long bolt, optionally secured with a first nut. The opposite end of the long screw is inserted into the corresponding structural element of assemblable enclosure 504 and typically secured with a second nut. In another example, interconnecting parts may be an extended parallel coupler providing a firm coupling between of tubes or pipes. In yet other examples, interconnecting parts are strings, belts and/or bands, threaded into respective apertures, grommets or eyelets of toroidally shaped structure of suspendable hanger component 512 and the corresponding apertures, grommets or eyelets in assemblable enclosure 504.

In some embodiments, installation 500 comprises anaerobic digester 514. Anaerobic digester 514 is preferably made of at least one sheet of pliable material, defining an essentially cylindrically closed structure; thereby rendering anaerobic digester 514 pliable and collapsible. In some embodiments, anaerobic digester 514 is manufactured by welding of polymeric sheets. Therefore anaerobic digester 514 is capable of assuming a collapsed or folded conformation. In other embodiments, however, anaerobic digester 514 is manufactured by welding and/or gluing segments polymeric sheets. In yet other embodiments, anaerobic digester 514 is manufactured by a means of molding, such as vacuum molding or blow molding.

In some embodiments, installation 500 comprises a plurality of fasteners 516. The plurality of fasteners 516 is attachable to an upper exterior portion of anaerobic digester 514. In some embodiments, each one of the plurality of fasteners 516 comprises at least one first portion 518, firmly attachable to anaerobic digester 514, as well as at least one second portion 520 operationally connectable to suspendable hanger component 512.

In some embodiments, each one of the plurality of fasteners 516 is configured to assume an open configuration, in which each one of plurality of fasteners 516 is configured to receive and/or adjoin to an element or portion associated with suspendable hanger component 512, and a closed configuration, in which each one of the plurality of fasteners 516 is configured to secure the respective element or portion associated with suspendable hanger component 512, thereby rendering anaerobic digester 514 reversibly hangable from and/or readily connectable to suspendable hanger component 512.

In some examples, each one of the plurality of fasteners 516 in a non-limiting manner include: a hook and loop fastener, string, buckle, snap connector, clips, strap, belt, chain, grommets, eyelets, tabs, etc. The first portion of each one of plurality of fasteners 516 is optionally a double hook, grommet or eyelet firmly attachable to anaerobic digester 514, whereas the second portion operationally connectable to the first portion and suspendable hanger component 512 is optionally a string. Anaerobic digester 514 is optionally fastened at a plurality of fastening points by a single elongated string, which is wrapped around suspendable hanger component 512 and/or threaded into suspendable hanger component 512. In some examples fasteners 516 hook and loop fasteners commercialized under the tradename of Velcro (R).

In another example, each one of the plurality of fasteners 516 may be a series of double ring and strap fasteners for securing anaerobic digester 514 to suspendable hanger component 512. Each fastener includes a double ring and strap. Straps are secured to suspendable hanger component 512 while double ring firmly attachable to anaerobic digester 514.

In some examples, anaerobic digester 514 includes upper end which comprises a plurality of grommets, tabs or eyelets disposed therein. Upper grommets are preferably equidistantly spaced along the upper edge of anaerobic digester 514. Upper grommets are optionally secured by hooks or straps firmly attachable to anaerobic digester 514, when suspendable hanger component 512 is in turn suspended from cylindrically shaped assemblable enclosure 504.

In some embodiments, anaerobic digester 514 comprises fastener central 518. Centrical fastener 518 is attachable at the center of the top portion of anaerobic digester 514. Centrical fastener 518 comprises at least one first portion, firmly attachable to anaerobic digester 514. Centrical fastener 518 further comprises at least one second portion, operationally connectable to a top cover (not shown), covering cylindrically shaped assemblable enclosure 504.

In some embodiments, centrical fastener 518 is configured to assume an open configuration in which centrical fastener 518 is configured to receive a portion of the top cover of cylindrically shaped assemblable enclosure 504. Centrical fastener 518 is configured to assume a closed configuration in which centrical fastener 518 is configured to secure a portion of the cover, thereby rendering anaerobic digester 514 reversibly attachable to the portion of the top cover covering cylindrically shaped assemblable enclosure 504 and conferring a convex shape to the top face of anaerobic digester 514.

In other embodiments, centrical fastener 518 is configured to assume an open configuration, in which centrical fastener 518 is configured to receive a portion of ballast weight. Centrical fastener 518 is configured to assume a closed configuration in which centrical fastener 518 is configured to secure a portion of the ballast weight, thereby rendering anaerobic digester 514 reversibly attachable to the ballast weight and conferring a concave shape to the top face of anaerobic digester 514.

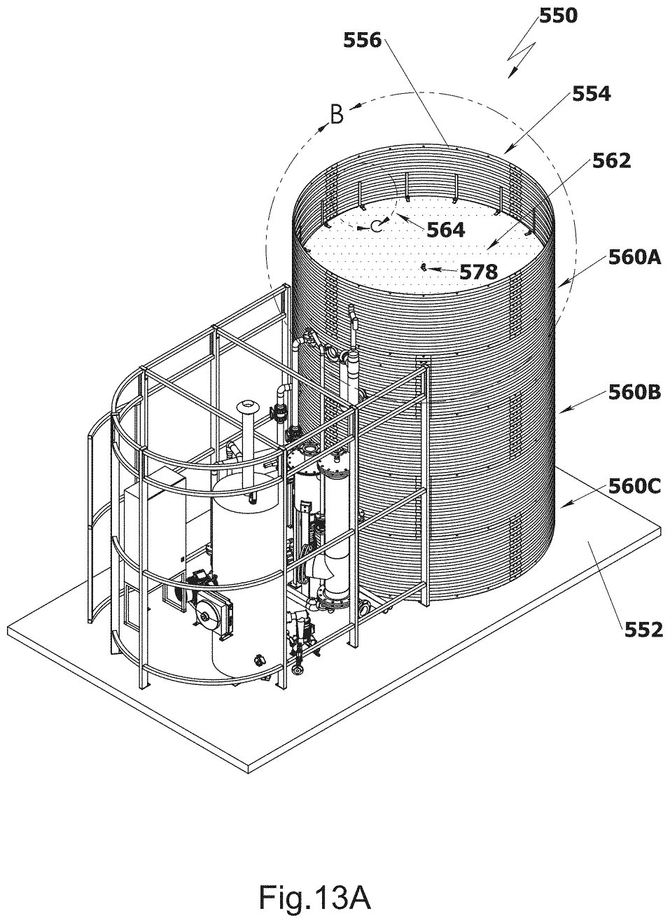

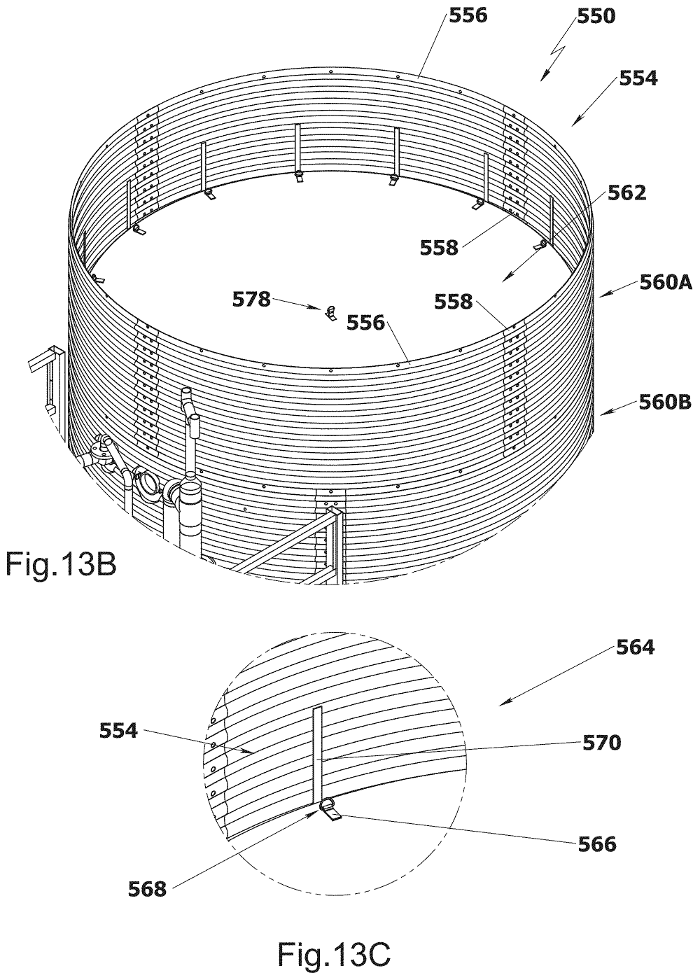

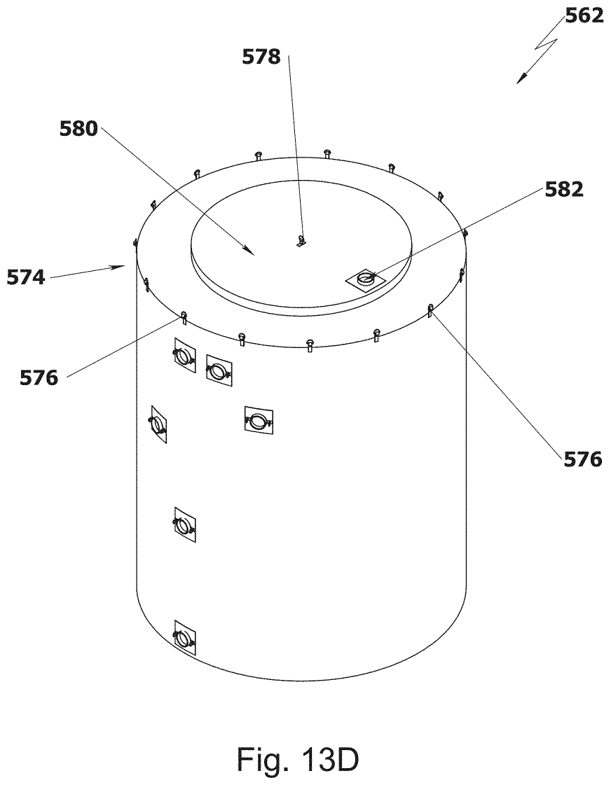

In accordance with another preferred embodiments of the present invention, reference is now made to FIG. 13A to 13C, showing an isometric view of a readily erectable installation 550 for recycling organic waste on an industrial scale without a hanger component, as well as a couple of enlarged views thereof. Readily erectable installation 550 of the embodiment of FIG. 13A to 13C illustrates various features that may be interchangeable with elements of any other embodiment described in the specification. Readily erectable installation 550 is mountable onto pedestal 552.

Installation 550 comprises pedestal 552, whereon readily erectable installation 550 is mountable. In some examples, readily erectable installation 550 is installable on existing floor, having essentially flat top horizontal surface, as pedestal 552. In some embodiments, installation 550 comprises cylindrically shaped assemblable enclosure 554, attachable to pedestal 552.

In some embodiments, cylindrically shaped assemblable enclosure 554 comprises a plurality of annular members 560A, 560B and 560C, assemblable in tandem along the longitudinal centerline of assemblable enclosure 554. In some examples, each pair of adjacent annular members, are respectively connected to each other by plurality of annular member connectors.

In some embodiments, each one of the plurality of annular members 560A, 560B and 560C comprises a sub-assembly of arcuate segments 556 connected by arcuate segments connectors 558. In some embodiments, installation 550 comprises anaerobic digester 562. Anaerobic digester 562 is essentially similar to digester 514.

In some embodiments, installation 550 comprises a plurality of fasteners 564. The plurality of fasteners 564 is attachable to an upper exterior portion of anaerobic digester 562. In some embodiments, each one of the plurality of fasteners 564 comprises at least one first portion 566, firmly attachable to anaerobic digester 562, as well as at least one second portion 570 operationally connectable to assemblable enclosure 554.

In some embodiments, each one of the plurality of fasteners 564 is configured to assume an open configuration, in which each one of plurality of fasteners 564 is configured to receive and/or adjoin to an element or portion associated with assemblable enclosure 554, and a closed configuration, in which each one of the plurality of fasteners 564 is configured to secure the respective element or portion associated with assemblable enclosure 554, thereby rendering anaerobic digester 562 reversibly hangable from and/or readily connectable to assemblable enclosure 554.

In some examples, each one of the plurality of fasteners 564 in a non-limiting manner include: a hook and loop fastener, string, buckle, snap connector, clips, strap, belt, chain, grommets, eyelets, tabs, etc. In some examples, each one of plurality of fasteners 564 optionally further comprises an interconnecting element 568, such as a double ring, hook, grommet or eyelet firmly attachable to at least one first portion 566 of fasteners 564, which is in turn further firmly attachable to anaerobic digester 562, whereas second portion 570 operationally connectable to assemblable enclosure 554. Anaerobic digester 562 is optionally fastened at a plurality of fastening points by a single elongated string, which is wrapped around assemblable enclosure 554 and/or threaded into to assemblable enclosure 554. In some examples fasteners 564 comprise hook and loop fasteners commercialized under the tradename of Velcro (R).

In another example, each one of the plurality of fasteners 564 may include a series of double ring elements 568 for securing to at least one first portion 566 firmly attachable to anaerobic digester 562, to straps 570 of fasteners 564 that are fastened to assemblable enclosure 554. Each fastener 564 optionally includes a double ring 568, strap 570 and strap 566. Straps 570 are secured to assemblable enclosure 554 while double ring 568 firmly attachable strap 566 which is in turn firmly attachable to anaerobic digester 562.

In accordance with another preferred embodiments of the present invention, reference is now made to FIG. 13D, showing an isometric view of anaerobic digester 562. In some embodiments, anaerobic digester 562 comprises upper end 574 which comprises plurality of fasteners 576. In some examples, plurality of fasteners 576 are grommets, tabs or eyelets. Plurality of fasteners 576 are preferably equidistantly spaced along upper end 574 of anaerobic digester 562. Plurality of fasteners 576 are optionally secured by hooks or straps firmly attachable to anaerobic digester 562, suspended from cylindrically shaped assemblable enclosure 554 shown in FIG. 13A to 13C.

In some embodiments, anaerobic digester 562 comprises central fastener 578. Centrical fastener 578 is attachable at the center of the top portion of anaerobic digester 562. In some embodiments, anaerobic digester 562 further comprises fluidic ballast weight 580. Centrical fastener 578 is configured to receive a portion of fluidic ballast weight 580. Centrical fastener 578 is optionally configured to assume a closed configuration in which centrical fastener 578 is configured to secure a portion of fluidic ballast weight 580, thereby rendering anaerobic digester 562 reversibly attachable to fluidic ballast weight 580.

In some embodiments, fluidic ballast weight 580 is centrally attached to the top face of anaerobic digester 562. Fluidic ballast weight 580 is configured for exerting gravitational force onto anaerobic digester 562, thereby forming positive gas pressure within anaerobic digester 562. In some embodiments, fluidic ballast weight 580 comprises inlet 582. Inlet 582 is configured for introducing water, sand or some other fluid into fluidic ballast weight 580, so that the former fills up the latter and the latter forms positive gas pressure within anaerobic digester 562.

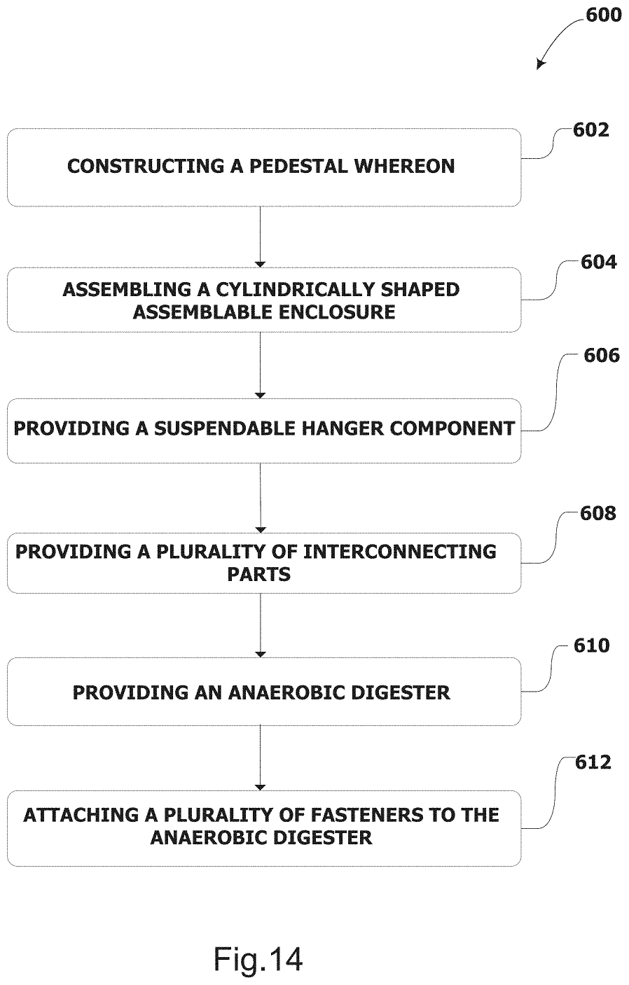

In accordance with some preferred embodiments of the present invention, reference is now made to FIG. 14, showing a flowchart of an embodiment of method 600 of readily erecting an installation for recycling organic waste on an industrial scale. Embodiment of method 600 shown in FIG. 15 illustrates various features that may be interchangeable with elements and/or features of any other embodiment described in the specification.

In some embodiments, method 600 commences with step 602 of constructing a pedestal whereon the installation is mountable. In some examples, step 602 is achievable by constructing a concrete or wooden platform forming a horizontal surface as a support for the mounting of the installation.

In some embodiments, method 600 proceeds to step 604 of assembling a cylindrically shaped assemblable enclosure. Step 604 of assembling a cylindrically shaped assemblable enclosure preferably includes the sub-steps of providing a plurality of arcuate segments, providing a plurality of arcuate segments connectors, assembling a plurality of annular member connectors by connecting the arcuate segments circumferentially in tandem around a longitudinal centerline of the cylindrically shaped assemblable enclosure of the installation, and connecting the annular members in tandem along the longitudinal centerline of an assemblable enclosure.

In some embodiments, method 600 further includes step 606 of providing a suspendable hanger component. In some embodiments, step 606 is achievable by providing a toroidally shaped structure, as well as by providing and/or forming a plurality of structural elements associated with the toroidally shaped structure of suspendable hanger component.

In some embodiments, method 600 yet further includes step 608 of providing and/or deploying a plurality of interconnecting parts. Step 608 of providing and/or deploying a plurality of interconnecting parts is configured for interconnecting the suspendable hanger component to the cylindrically shaped assemblable enclosure.

In some embodiments, method 600 still includes step 610 of providing and/or deploying an anaerobic digester. In some examples, step 610 of providing an anaerobic digester is preferably providing at least one sheet of pliable material, defining an essentially cylindrically closed structure; thereby rendering anaerobic digester pliable and collapsible.

In some embodiments, method 600 yet still includes step 612 of attaching to an upper exterior portion of the anaerobic digester. In some embodiments, step 612 is achievable by attaching at least one first portion of the plurality of the fasteners to the anaerobic digester and/or connecting at least one second portion of the plurality of the fasteners to the suspendable hanger component.

In some embodiments, step 612 of attaching a plurality of fasteners further comprises at least some of the sub-steps of providing the plurality of the fasteners in an open conformation configured for receiving a structural element associated with the suspendable hanger component and altering the plurality of the fasteners into a closed conformation configured for securing the structural element associated with the suspendable hanger component, wherein altering the plurality of the fasteners between the open and closed conformations renders the anaerobic digester reversibly attachable to the suspendable hanger component.

In some embodiments, method 600 yet still includes step 612 of attaching to an upper exterior portion of the anaerobic digester. In some embodiments, step 612 is achievable by attaching at least one first portion of the plurality of the fasteners to the anaerobic digester and/or connecting at least one second portion of the plurality of the fasteners to the cylindrically shaped assemblable enclosure.

In some embodiments, step 612 of attaching a plurality of fasteners further comprises at least some of the sub-steps of providing the plurality of the fasteners in an open conformation configured for receiving the structural element associated with the cylindrically shaped assemblable enclosure and altering the plurality of the fasteners into a closed conformation configured for securing the structural element associated with the cylindrically shaped assemblable enclosure, wherein altering the plurality of the fasteners between the open and closed conformations renders the anaerobic digester reversibly attachable to the cylindrically shaped assemblable enclosure.

It will be appreciated by persons skilled in the art that the present invention is not limited by what has been particularly shown and described herein above. Rather the scope of the invention is defined by the claims which follow:

1. A readily erectable installation for recycling organic waste on an industrial scale, said appliance comprises:
(a) a pedestal whereon said readily erectable installation is mountable;
(b) a cylindrically shaped assemblable enclosure comprising:
(I) a plurality of arcuate segments circumferentially assemblable in tandem around a longitudinal centerline of said assemblable enclosure;
(II) a plurality of arcuate segment connectors, configured to connect said arcuate segments into an annular shape;
(III) a plurality of annular members, wherein each one said annular members comprising a sub-assembly of said arcuate segments and said arcuate segment connectors, said annular members are assemblable in tandem along said longitudinal centerline of said assemblable enclosure;
(IV) a plurality of annular member connectors, configured to connect said annular members to form a cylindrical shape of said assemblable enclosure;
(c) a suspendable hanger component comprising:
(I) a toroidally shaped structure;
(II) a plurality of structural elements, disposed on said toroidally shaped structure, configured for attachment of said toroidally shaped structure to interconnecting parts;
(d) a plurality of interconnecting parts, said interconnecting parts are configured to interconnect between said cylindrically shaped assemblable enclosure and said suspendable hanger component;
(e) an anaerobic digester comprising a sheet of pliable material, forming an essentially cylindrically closed structure;
(f) a plurality of fasteners attachable to an upper exterior portion of said anaerobic digester;
wherein each one of said plurality of fasteners comprising:
(I) at least one first portion, firmly attachable to said anaerobic digester;
(II) at least one second portion, operationally connectable to said first portion and said suspendable hanger component;
wherein each one of said plurality of fasteners is configured to assume:
(i) an open configuration, in which said portions are configured to receive said suspendable hanger component, and
(ii) a closed configuration, in which said portions are configured to secure said suspendable hanger component;
thereby rendering said anaerobic digester readily attachable to said suspendable hanger component.

2. The readily erectable installation as in claim 1, wherein said pedestal is a platform.

3. The readily erectable installation as in claim 1, wherein said toroidally shaped structure is toroidally shaped shell.

4. The readily erectable installation as in claim 1, wherein said toroidally shaped structure is a curved tube which closes in on itself.

5. The readily erectable installation as in claim 1, wherein said arcuate segments are arcuate corrugated metal sheet slabs.

6. The readily erectable installation as in claim 1, wherein said interconnecting parts are selected from the group consisting of: a screw, bolt and nut, extended parallel coupler, fixing clamper, string, band, belt, loop and chain.

7. The readily erectable installation as in claim 1, wherein said fastener is selected from the group consisting of: a hook and loop fastener, string, buckle, snap connector, clip, strap, belt, chain, grommet, eyelet and tab.

8. The readily erectable installation as in claim 1, wherein said anaerobic digester is fastened in a plurality of fastening points by a string.

9. The readily erectable installation as in claim 1, wherein said plurality of said annular members, is pre-assembled from said plurality of said arcuate segments and said arcuate segment connectors, prior to assembling said plurality of said annular members in tandem along said longitudinal centerline of said assemblable enclosure.

10. A method of readily erecting an installation for recycling organic waste on an industrial scale, said appliance comprises:
(a) constructing a pedestal whereon said installation is mountable;
(b) assembling a cylindrically shaped assemblable enclosure comprising:
(I) providing a plurality of arcuate segments;
(II) providing a plurality of arcuate segment connectors;
(III) assembling a plurality of annular member connectors by connecting said arcuate segments circumferentially in tandem around a longitudinal centerline of said cylindrically shaped assemblable enclosure;
(IV) connecting said annular members in tandem around a longitudinal centerline of an assemblable enclosure;
(c) providing a suspendable hanger component comprising:
(I) a toroidally shaped structure;
(II) a plurality of structural elements, wherein said toroidally shaped structure is disposed;
(d) providing a plurality of interconnecting parts;
(e) providing an anaerobic digester forming an essentially cylindrically closed structure;
(f) attaching a plurality of fasteners to an upper exterior portion of said anaerobic digester;
wherein attaching each one of said plurality of fasteners comprising:
(I) attaching at least one first portion to said anaerobic digester;
(II) connecting at least one second portion to said first portion and said suspendable hanger component;
wherein attaching each one of said plurality of fasteners comprising:
(i) receiving said suspendable hanger component in an open conformation, and
(ii) securing said suspendable hanger component providing in a closed conformation;
wherein switching between said open and closed configurations renders said anaerobic digester readily attachable to said suspendable hanger component.

11. The method of readily erecting an installation for recycling organic waste as in claim 10, wherein said pedestal is a platform.

12. The method of readily erecting an installation for recycling organic waste as in claim 10, wherein said toroidally shaped structure is toroidally shaped shell.

13. The method of readily erecting an installation for recycling organic waste as in claim 10, wherein said toroidally shaped structure is a curved tube which closes in on itself.

14. The method of readily erecting an installation for recycling organic waste as in claim 10, wherein said arcuate segments are arcuate corrugated metal sheet slabs.

15. The method of readily erecting an installation for recycling organic waste as in claim 10, wherein said interconnecting parts are selected from the group consisting of: a screw, bolt and nut, extended parallel coupler, fixing clamper, string, band, belt, loop and chain.

16. The method of readily erecting an installation for recycling organic waste as in claim 10, wherein said fastener is selected from the group consisting of: a hook and loop fastener, string, buckle, snap connector, clip, strap, belt, chain, grommet, eyelet and tab.

17. The method of readily erecting an installation for recycling organic waste as in claim 10, wherein said anaerobic digester is fastened in a plurality of fastening points by a string.

18. The method of readily erecting an installation for recycling organic waste as in claim 10, wherein said plurality of said annular members, is pre-assembled from said plurality of said arcuate segments and said arcuate segment connectors, prior to assembling said plurality of said annular members in tandem along said longitudinal centerline of said assemblable enclosure.

19. A readily erectable installation for recycling organic waste on an industrial scale, said appliance comprises:
(a) a pedestal whereon said readily erectable installation is mountable;
(b) a cylindrically shaped assemblable enclosure comprising:
(I) a plurality of arcuate segments circumferentially assemblable in tandem around a longitudinal centerline of said assemblable enclosure;
(II) a plurality of arcuate segment connectors, configured to connect said arcuate segments into an annular shape;
(III) a plurality of annular members, wherein each one said annular members comprising a sub-assembly of said arcuate segments and said arcuate segment connectors, said annular members are assemblable in tandem along said longitudinal centerline of said assemblable enclosure;
(IV) a plurality of annular member connectors, configured to connect said annular members to form a cylindrical shape of said assemblable enclosure;
(c) an anaerobic digester comprising a sheet of pliable material, forming an essentially cylindrically closed structure;
wherein said anaerobic digester is accommodatable within said cylindrically shaped assemblable enclosure.

20. The readily erectable installation as in claim 19, wherein said pedestal is a platform.

21. The readily erectable installation as in claim 19, wherein said arcuate segments are arcuate corrugated metal sheet slabs.

22. The readily erectable installation as in claim 19, further comprises a plurality of fasteners attachable to an upper exterior portion of said anaerobic digester;
wherein each one of said plurality of fasteners comprising:
(I) at least one first portion, attachable to said anaerobic digester;
(II) at least one second portion, operationally connectable to said first portion and a top portion of said cylindrically shaped assemblable enclosure;
wherein each one of said plurality of fasteners is configured to assume:
(i) an open conformation, in which said fastener is configured to receive said top portion of said cylindrically shaped assemblable enclosure, and
(ii) a closed conformation, in which said fastener is configured to secure said top portion of said cylindrically shaped assemblable enclosure; thereby rendering said anaerobic digester readily attachable to said top portion of said cylindrically shaped assemblable enclosure.

23. The readily erectable installation as in claim 22, wherein said fastener is selected from the group consisting of: a hook and loop fastener, string, buckle, snap connector, clip, strap, belt, chain, grommet, eyelet and tab.

24. The readily erectable installation as in claim 19, wherein said plurality of said annular members, is pre-assembled from said plurality of said arcuate segments and said arcuate segment connectors, prior to assembling said plurality of said annular members in tandem along said longitudinal centerline of said assemblable enclosure.

25. A method of readily erecting an installation for recycling organic waste on an industrial scale, said appliance comprises:
(a) constructing a pedestal whereon said installation is mountable;
(b) assembling a cylindrically shaped assemblable enclosure comprising:
(I) providing a plurality of arcuate segments;
(II) providing a plurality of arcuate segment connectors;
(III) assembling a plurality of annular member connectors by connecting said arcuate segments circumferentially in tandem around a longitudinal centerline of said cylindrically shaped assemblable enclosure;
(IV) connecting said annular members in tandem around a longitudinal centerline of an assemblable enclosure;
(c) providing an anaerobic digester forming an essentially cylindrically closed structure;
(d) attaching a plurality of fasteners to an upper exterior portion of said anaerobic digester;
wherein attaching each one of said plurality of fasteners comprising:
(I) attaching at least one first portion to said anaerobic digester;
(II) connecting at least one second portion to said first portion and said cylindrically shaped assemblable enclosure;
wherein attaching each one of said plurality of fasteners comprising:
(i) receiving said cylindrically shaped assemblable enclosure in an open conformation for, and
(ii) securing said cylindrically shaped assemblable enclosure in a closed conformation;
wherein switching between said open and closed configurations renders said anaerobic digester readily attachable to said cylindrically shaped assemblable enclosure.

26. The readily erectable installation as in claim 25, wherein said pedestal is a platform.

27. The readily erectable installation as in claim 25, wherein said arcuate segments are arcuate corrugated metal sheet slabs.

28. The readily erectable installation as in claim 25, wherein said fastener is selected from the group consisting of: a hook and loop fastener, string, buckle, snap connector, clip, strap, belt, chain, grommet, eyelet and tab.

29. The readily erectable installation as in claim 25, wherein said anaerobic digester is fastened in a plurality of fastening points by a string.
